SEQUENCE LISTING (49 sequences)

SEQ ID NO: 1 - protein, 550 aa, synthetic construct
MHPQQLVISWFSLVLLASPLMAIWELEKDVYVVELDWHPDAPGEMVVLTCHTPEEDDITWTSAQSSEVLGSGKTLTIQVKEFGDAGQYTCHKGGKVLSRSLLLIHKKEDGIWSTDILKEQKESKNKIFLKCEAKNYSGRFTCWWLTAISTDLKFSVKSSRGFSDPQGVTCGAVTLSAERVRVDNRDYKKYTVECQEGSACPSAEESLPIEVVVDAIHKLKYENYTSSFFIRDIIKPDPPTNLQLKPLKNSRHVEVSWEYPDTWSTPHSYFSLTFCIQAQGKNNREKKDRLCVDKTSAKVVCHKDAKIRVQARDRYYSSSWSDWASVSCSGSGSSRGGSGSGGSGGGGSKLRAVPEGSSPAWIQGQQLSQKLCTLVWSAHPPMGHVDLPREEGDGETTSDVPRIQCGDGCDPQGLKDNSQFCLQRIHQGLVFYEKLLGSDIFTGEPSLLPDGPVGQLHANLLGLRQLLQPEGHHWETEQTPSPSPSQPWQRLLLRLKILRSLQAFVAVAARVFAHGAATLSPGSGSGLNDIFEAQKIEWHEHHHHHHHHHH

SEQ ID NO: 2 - protein, 550 aa, synthetic construct
MHPQQLVIAWFSLVLLAPPLMAIWELEKNVYVVELDWHPDAPGEMVVLTCDTPEEDDITWTSDQSSEVLGSGKTLTIQVKEFADAGQYTCHKGGEVLSHSFLLIHKKEDGIWSTDILREQKESKNKIFLKCEAKNYSGRFTCWWLTAISTDLKFTVKSSRGSSDPQGVTCGAATLSAEKVRVDNRDYKKYTVECQEGSACPAAEESLPIEVVVDAIHKLKYENYTSSFFIRDIIKPDPPKNLQLKPLKNSRHVEVSWEYPDTWSTPHSYFSLTFGVQVQGKNNREKKDRLSVDKTSAKVVCHKDAKIRVQARDRYYSSSWSNWASVSCSGSGSSRGGSGSGGSGGGGSKLRAVPEGSSPAWALGQQLSQKLCTLAWSAHPPTGHVDLPREQGDDETTDDVPHIQCGDGCDPQGLRDNSQFCLQRIHQGLVFYEKLLGSDIFTGEPSLLPDSPVGQLHASLLGLRQLLQPDGQHWETEQTPNPSPNQPWQRLLLRFKILRSLQAFVAVAARVFAHGAATLSPGSGSGLNDIFEAQKIEWHEHHHHHHHHHH

SEQ ID NO: 3 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 4 - protein, 11 aa, synthetic construct
RASQGISSWLA

SEQ ID NO: 5 - protein, 8 aa, synthetic construct
YAASSLQS

SEQ ID NO: 6 - protein, 9 aa, synthetic construct
QQYNIYPYT

SEQ ID NO: 7 - protein, 119 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 8 - protein, 13 aa, synthetic construct
KGSGYSFTTYWLG

SEQ ID NO: 9 - protein, 10 aa, synthetic construct
IMSPVDSDIR

SEQ ID NO: 10 - protein, 12 aa, synthetic construct
ARRRPGQGYFDF

SEQ ID NO: 11 - protein, 107 aa, synthetic construct
DIVMTQTPLSLSVSPGEPASISCRASQGISSWLAWYQQKAEKAPKSLIYAASSLQSGVPDRFSGSGSGTDFTLRIGRVEAEDAGIYYCQQYNIYPYTFGQGTRLEVR

SEQ ID NO: 12 - protein, 119 aa, synthetic construct
EVQLVQSGAEVKKPGASVKVSCKGSGYSFTTYWLGWVRQAPGAGLDWMGIMSPVDSDIRYSPSFQGQVTMSVDTSTSTAYMELSSLRAGDIAVYYCARRRPGQGYFDFWGQGTLVTVSS

SEQ ID NO: 13 - protein, 107 aa, synthetic construct
DIVMTQSPASLSLSQEEKVTITCRASQGISSWLAWYQQKPEKAPKSLIYAASSLQSGVPSRFSGSGSGTDFSFTISSLEPEDVAVYYCQQYNIYPYTFGQGTKLEIK

SEQ ID NO: 14 - protein, 119 aa, synthetic construct
EVQLVESGGDLVKPGGSLRLSCKGSGYSFTTYWLGWVRQMPGKGLDWIGIMSPVDSDIRYSPSFQGRFTISRDNAKNTVYLQMNSLRAEDTAMYYCARRRPGQGYFDFWGQGTLVTVSS

SEQ ID NO: 15 - protein, 107 aa, synthetic construct
EIQMTQSPSSLSASPGDRVTITCRASQGISSWLAWYQQKPEKAPKSLIYAASSLQSGVPSRFSGSGSGTDFTLTISSLEPEDAATYYCQQYNIYPYTFGQGTKLEIK

SEQ ID NO: 16 - protein, 119 aa, synthetic construct
QVLLVQSGAEVRKPEASVKIFCKGSGYSFTTYWLGWVRQAPGQGLDWMGIMSPVDSDIRYAQNFQGKLTLTVDTSTNTAYMELRNLRSTDTAVYYCARRRPGQGYFDFWGQGTLVTVSS

SEQ ID NO: 17 - protein, 217 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 18 - protein, 454 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 19 - protein, 217 aa, synthetic construct
DIVMTQTPLSLSVSPGEPASISCRASQGISSWLAWYQQKAEKAPKSLIYAASSLQSGVPDRFSGSGSGTDFTLRIGRVEAEDAGIYYCQQYNIYPYTFGQGTRLEVRRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 20 - protein, 454 aa, synthetic construct
EVQLVQSGAEVKKPGASVKVSCKGSGYSFTTYWLGWVRQAPGAGLDWMGIMSPVDSDIRYSPSFQGQVTMSVDTSTSTAYMELSSLRAGDIAVYYCARRRPGQGYFDFWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 21 - protein, 454 aa, synthetic construct
EVQLVQSGAEVKKPGASVKVSCKGSGYSFTTYWLGWVRQAPGAGLDWMGIMSPVDSDIRYSPSFQGQVTMSVDTSTSTAYMELSSLRAGDIAVYYCARRRPGQGYFDFWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLWCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 22 - protein, 217 aa, synthetic construct
DIVMTQSPASLSLSQEEKVTITCRASQGISSWLAWYQQKPEKAPKSLIYAASSLQSGVPSRFSGSGSGTDFSFTISSLEPEDVAVYYCQQYNIYPYTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 23 - protein, 454 aa, synthetic construct
EVQLVESGGDLVKPGGSLRLSCKGSGYSFTTYWLGWVRQMPGKGLDWIGIMSPVDSDIRYSPSFQGRFTISRDNAKNTVYLQMNSLRAEDTAMYYCARRRPGQGYFDFWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 24 - protein, 217 aa, synthetic construct
EIQMTQSPSSLSASPGDRVTITCRASQGISSWLAWYQQKPEKAPKSLIYAASSLQSGVPSRFSGSGSGTDFTLTISSLEPEDAATYYCQQYNIYPYTFGQGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 25 - protein, 454 aa, synthetic construct
QVLLVQSGAEVRKPEASVKIFCKGSGYSFTTYWLGWVRQAPGQGLDWMGIMSPVDSDIRYAQNFQGKLTLTVDTSTNTAYMELRNLRSTDTAVYYCARRRPGQGYFDFWGQGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLYISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 26 - protein, 107 aa, synthetic construct [sequence omitted]

SEQ ID NO: 27 - protein, 11 aa, synthetic construct
RASQGIRNYLA

SEQ ID NO: 28 - protein, 6 aa, synthetic construct
AASTLQ

SEQ ID NO: 29 - protein, 9 aa, synthetic construct
QRYNRAPYT

SEQ ID NO: 30 - protein, 121 aa, synthetic construct [sequence omitted]

SEQ ID NO: 31 - protein, 9 aa, synthetic construct
FTFDDYAMH

SEQ ID NO: 32 - protein, 18 aa, synthetic construct
AITWNSGHIDYADSVEGR

SEQ ID NO: 33 - protein, 14 aa, synthetic construct
AKVSYLSTASSLDY

SEQ ID NO: 34 - protein, 107 aa, synthetic construct
DIVMTQTPLSLSVSPGEPASISCRASQGIRNYLAWYQQKPGKAPKLLIYAASTLQSGVPDRFSGSGSGTDFTLRIGRVEAEDAGIYYCQRYNRAPYTFGQGTRLEVR

SEQ ID NO: 35 - protein, 121 aa, synthetic construct
EVQLVESGGDLVKPAESLRLSCVASGFTFDDYAMHWVRQAPGKGLEWVSAITWNSGHIDYADSVEGRFTISRDNARNTLYLQMNSLRAEDTAVYYCAKVSYLSTASSLDYWGQGTLVTVSS

SEQ ID NO: 36 - protein, 107 aa, synthetic construct
DVVMTQTPLSLAVTPGEPASISCRASQGIRNYLAWYQQKPGKAPKLLIYAASTLQSGVPDRFSGSGSGTDFTLRISRVEADDVGVYYCQRYNRAPYTFGQGTKLEIR

SEQ ID NO: 37 - protein, 121 aa, synthetic construct
DVQLVESGGDLVKPGGSQRLTCVASGFTFDDYAMHWVRQAPGKGLEWVSAITWNSGHIDYADSVEGRFTISRDNAKNTLYLQMNSLRTEDTATYYCAKVSYLSTASSLDYWGQGALVTVSS

SEQ ID NO: 38 - protein, 217 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 39 - protein, 456 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 40 - protein, 217 aa, synthetic construct
DIVMTQTPLSLSVSPGEPASISCRASQGIRNYLAWYQQKPGKAPKLLIYAASTLQSGVPDRFSGSGSGTDFTLRIGRVEAEDAGIYYCQRYNRAPYTFGQGTRLEVRRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 41 - protein, 217 aa, synthetic construct
DIVMTQTPLSLSVSPGEPASISCRASQGIRNYLAWYQQKPGCAPKLLIYAASTLQSGVPDRFSGSGSGTDFTLRIGRVEAEDAGIYYCQRYNRAPYTFGCGTRLEVRRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSESQRVD

SEQ ID NO: 42 - protein, 456 aa, synthetic construct
EVQLVESGGDLVKPAESLRLSCVASGFTFDDYAMHWVRQAPGKGLEWVSAITWNSGHIDYADSVEGRFTISRDNARNTLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 43 - protein, 456 aa, synthetic construct
EVQLVESGGDLVKPAESLRLSCVASGFTFDDYAMHWVRQAPGKGLEWVSAITWNSGHIDYADSVEGRFTISRDNARNTLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 44 - protein, 455 aa, synthetic construct
EVQLVESGGDLVKPAESLRLSCVASGFTFDDYAMHWVRQAPGKCLEWVSAITWNSGHIDYADSVEGRFTISRDNARNTLYLQMNSLRAEDTAVYYCAKVSYLSTASSLDYWGQGTLVTVSSASTTAPSVFPLAPSSGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLSCAIKDFFPPDIDVEWQSNGQQEPESKYRTTPPQLDEDGSYFLASKLSVDKSRWQRGDTFICAVMHEALHNHYTQESLSHSPG

SEQ ID NO: 45 - protein, 245 aa, synthetic construct
EVQLVESGGDLVKPAESLRLSCVASGFTFDDYAMHWVRQAPGKGLEWVSAITWNSGHIDYADSVEGRFTISRDNARNTLYLQMNSLRAEDTAVYYCAKVSYLSTASSLDYWGQGTLVTVSSGGGGGSGGGGSGGGGGSDIVMTQTPLSLSVSPGEPASISCRASQGIRNYLAWYQQKPGKAPKLLIYAASTLQSGVPDRFSGSGSGTDFTLRIGRVEAEDAGIYYCQRYNRAPYTFGQGTRLEVR

SEQ ID NO: 46 - protein, 245 aa, synthetic construct
EVQLVESGGDLVKPAESLRLSCVASGFTFDDYAMHWVRQAPGKCLEWVSAITWNSGHIDYADSVEGRFTISRDNARNTLYLQMNSLRAEDTAVYYCAKVSYLSTASSLDYWGQGTLVTVSSGGGGGSGGGGSGGGGGSDIVMTQTPLSLSVSPGEPASISCRASQGIRNYLAWYQQKPGKAPKLLIYAASTLQSGVPDRFSGSGSGTDFTLRIGRVEAEDAGIYYCQRYNRAPYTFGCGTRLEVR

SEQ ID NO: 47 - protein, 706 aa, synthetic construct
EVQLVQSGAEVKKPGASVKVSCKGSGYSFTTYWLGWVRQAPGAGLDWMGIMSPVDSDIRYSPSFQGQVTMSVDTSTSTAYMELSSLRAGDIAVYYCARRRPGQGYFDFWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGGGGGSGGGEVQLVESGGDLVKPAESLRLSCVASGFTFDDYAMHWVRQAPGKGLEWVSAITWNSGHIDYADSVEGRFTISRDNARNTLYLQMNSLRAEDTAVYYCAKVSYLSTASSLDYWGQGTLVTVSSGGGGGSGGGGSGGGGGSDIVMTQTPLSLSVSPGEPASISCRASQGIRNYLAWYQQKPGKAPKLLIYAASTLQSGVPDRFSGSGSGTDFTLRIGRVEAEDAGIYYCQRYNRAPYTFGQGTRLEVR

SEQ ID NO: 48 - protein, 706 aa, synthetic construct
EVQLVQSGAEVKKPGASVKVSCKGSGYSFTTYWLGWVRQAPGAGLDWMGIMSPVDSDIRYSPSFQGQVTMSVDTSTSTAYMELSSLRAGDIAVYYCARRRPGQGYFDFWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGGGGGSGGGEVQLVESGGDLVKPAESLRLSCVASGFTFDDYAMHWVRQAPGKCLEWVSAITWNSGHIDYADSVEGRFTISRDNARNTLYLQMNSLRAEDTAVYYCAKVSYLSTASSLDYWGQGTLVTVSSGGGGGSGGGGSGGGGGSDIVMTQTPLSLSVSPGEPASISCRASQGIRNYLAWYQQKPGKAPKLLIYAASTLQSGVPDRFSGSGSGTDFTLRIGRVEAEDAGIYYCQRYNRAPYTFGCGTRLEVR

SEQ ID NO: 49 - protein, 17 aa, synthetic construct
GGGGGSGGGGSGGGGGS

(19) United States
(12) Patent Application Publication
(10) Pub. No.: US 2026/0209333 A1
(43) Pub. Date: Jul. 23, 2026

(54) ANTI-IL23 AND ANTI-TNFa ANTIBODIES: COMPOSITIONS AND VETERINARY USE

(71) Applicant: Vetmab Biosciences, Inc., San Diego, CA (US)

(72) Inventor: Hangjun ZHAN, San Diego, CA (US)

(73) Assignee: Vetmab Biosciences, Inc., San Diego, CA (US)

(21) Appl. No.: 19/138,328

(22) PCT Filed: Dec. 14, 2023
(86) PCT No.: PCT/US2023/084003

§ 371 Date: Jun. 12, 2025

Related U.S. Application Data
(60) Provisional application No. 63/387,777, filed on Dec. 16, 2022.

(51) Int. Cl. C07K 16/24; A61K 39/00; A61K 45/06; G01N 33/68

(52) U.S. Cl. CPC C07K 16/244; A61K 45/06; C07K 16/241; G01N 33/6863; G01N 33/6869; A61K 2039/54; A61K 2039/545; C07K 2317/14; C07K 2317/24; C07K 2317/31; C07K 2317/33; C07K 2317/56; C07K 2317/622; C07K 2317/76; G01N 2333/525; G01N 2333/54

(57) ABSTRACT

Provided are various embodiments relating to caninized, felinized, or equinized antibodies that bind canine, feline, and/or equine IL23 and/or TNFα, including bispecific antibodies that bind to both IL23 and TNFα. Such antibodies can be used alone or in combination in methods to treat canine, feline, and/or equine subjects with inflammatory conditions, such as inflammatory conditions in canines and felines, such as inflammatory bowel disease (IBD), osteoarthritis, and gastroenteritis.

### CROSS REFERENCE TO RELATED APPLICATIONS

This application is a U.S. national application filed under 35 U.S.C. 371 of International Patent Application No. PCT/US2023/084003, filed Dec. 14, 2023, which claims the priority benefit of U.S. Provisional Patent Application No. 63/387,777, filed on Dec. 16, 2022, the entirety of each of which is incorporated herein by reference.

### FIELD

This disclosure relates to caninized, felinized, and equinized anti-IL23 and anti-TNFα antibodies, and their uses, for example, use for treating canine, feline, or equine IL23-mediated and/or TNFα-mediated disorders, such as chronic enteropathy-inflammatory bowel disease (IBD), psoriasis, rheumatoid arthritis, osteoarthritis, sepsis, and multiple sclerosis (MS).

### REFERENCE TO SEQUENCE LISTING

The official copy of the Sequence Listing is submitted concurrently with the specification via USPTO Patent Center as an WIPO Standard ST.26 formatted XML file with file name “20136-001WOI.xml”, a creation date of Dec. 13, 2023, and a size of 53,419 bytes. This Sequence Listing filed via USPTO Patent Center is part of the specification and is incorporated in its entirety by reference herein.

### BACKGROUND

Interleukin 23 (IL23) is a heterodimeric cytokine composed of an IL-12B (IL-12p40) subunit (which is shared with IL-12) and an IL-23A (IL-23p19) subunit. IL23 is an inflammatory cytokine that is part of the IL-12 family of cytokines. It has been shown to be a key cytokine for T helper type 17 cell (Th17 cell) maintenance and expansion. IL23 is associated with a number of chronic inflammatory disorders in humans including diseases such as psoriasis, rheumatoid arthritis, psoriatic arthritis, osteoarthritis, multiple sclerosis, inflammatory bowel disease (IBD).

Like humans, companion animals such as cats, dogs, and horses, suffer from chronic inflammatory disorders, however, there has been no demonstration to date that anti IL23 could be used to treat such diseases in companion animals. Moreover, proteins with significant human-derived amino acid sequence content can be immunogenic in non-human animals and might not bind companion animal IL23 in a manner that provides an equally beneficial therapeutic effect in the companion animal. See Mauldin et al., August 2010, 21(4):373-382.

Inflammatory bowel disease (IBD) is a collective term used to describe disorders associated with chronic inflammation of the gastrointestinal tract. While the exact etiology of companion animal IBD remains unknown, alterations in immune system tolerance to dietary antigens, the intestinal microbiome and genetic susceptibility may all play a role. Canine, feline and equine IBD may share a similar etiology with the disorder that occurs in humans (also known as Crohn's disease and ulcerative colitis), but the clinical syndrome and histologic changes may be slightly different. Canine IBD is characterized by persistent or recurrent signs such as vomiting, diarrhea, abdominal pain, weight loss, and/or alterations in appetite, and inflammation in the GI tract. (Ettinger and Feldman; Suchodolski J S et al. “The fecal microbiome in dogs with acute diarrhea and idiopathic inflammatory bowel disease.” PLoS ONE. 2012; 7(12): e51907. doi:10.1371/journal.pone.0051907; Suchodolski J S. “Companion-animals symposium: Microbes and gastrointestinal health of dogs and cats.” J Anim Sci. 2010; 89(5):1520-1530. Feline IBD is characterized by vomiting, diarrhea and weight loss, along with inflammation in the GI tract, most commonly the small intestine (Jergens A. E. (2012). Feline idiopathic inflammatory bowel disease: what we know and what remains to be unraveled. Journal of feline medicine and surgery, 14(7), 445-458. https://doi.org/10.1177/1098612X12451548). Equine IBD is characterized by lethargy, diarrhea colic and weight loss (Boshuizen, B., et al. (2018). Inflammatory bowel disease (IBD) in horses: a retrospective study exploring the value of different diagnostic approaches. BMC veterinary research, 14(1), 21. https://doi.org/10.1186/s12917-018-1343-1).

Accurate diagnosis of companion animal IBD can be challenging and the term “idiopathic” is often used when an exact causative agent cannot be identified. A complete history and physical exam followed by laboratory investigation, diagnostic imaging, and intestinal biopsy (to demonstrate the presence of inflammation) are typically recommended. In canine, feline and equine IBD, the prognosis and response to traditional therapy varies and can range from moderate to poor. However, studies evaluating cytokines in companion animals with IBD have shown to have similar upregulation cytokines like IL-23 and TNFα as is seen in humans (Jergens, A. E., & Simpson, K. W. (2012). Inflammatory bowel disease in veterinary medicine. Frontiers in bioscience (Elite edition), 4(4), 1404-1419. https://doi.org/10.2741/e470; Jergens A. E. (2012). Feline idiopathic inflammatory bowel disease: what we know and what remains to be unraveled. Journal of feline medicine and surgery, 14(7), 445-458. https://doi.org/10.1177/1098612X12451548; Cerquetella, M., Spaterna, A., Laus, F., Tesei, B., Rossi, G., Antonelli, E., Villanacci, V., & Bassotti, G. (2010). Inflammatory bowel disease in the dog: differences and similarities with humans. World journal of gastroenterology, 16(9), 1050-1056. https://doi.org/10.3748/wig.v16.i9.1050; Olofsson, K. M., Hjertner, B., Fossum, C., Press, C. M., & Lindberg, R. (2015). Expression of T helper type 17 (Th17)-associated cytokines and toll-like receptor 4 and their correlation with Foxp3 positive cells in rectal biopsies of horses with clinical signs of inflammatory bowel disease. Veterinary journal (London, England: 1997), 206(1), 97-104. https://doi.org/10.1016/j.tvjl.2015.07.003). However, many of the often-prescribed biologics successfully used to treat IBD in humans are not available to companion animals, leaving limited treatments.

Gastroenteritis in humans and companion animals is associated with inflammation in the GI tract. Gastroenteritis in the canine and feline animals often presents with diarrhea and vomiting. Similar to IBD, studies evaluating gastroenteritis have found the cytokines involved in inflammation like IL-23 and TNFα are involved (Godinez, I., Keestra, A. M., Specs, A., & Bäumler, A. J. (2011). The IL-23 axis in Salmonella gastroenteritis. Cellular microbiology, 13(11), 1639-1647; Muñoz-Cruz, S., Gömez-Garcia, A., Millán-Ibarra, J., Giono-Cerezo, S., & Yëpez-Mulia, L. (2010). Giardia lamblia: interleukin 6 and tumor necrosis factor-alpha release from mast cells induced through an Ig-independent pathway. Experimental parasitology, 126(3), 298-303. https://doi.org/10.1016/i.exppara.2010.06.013) suggesting that treatments targeting IL-23 and TNFα cytokines could assist in treatment of gastroenteritis (Zhou, P., Li, E., SHEA-DONOHUE, T., & Singer, S. M. (2007). Tumour necrosis factor α contributes to protection against Giardia lamblia infection in mice. Parasite immunology, 29(7), 367-374). Current treatments for gastroenteritis in companion animals are limited to electrolytes and antibiotics and additional compounds addressing inflammation in the GI tract could be beneficial for treatment.

In neonate foals, sepsis is an inflammatory response syndrome (SIRS) that results from failure to passive transfer and foals present with symptoms including pneumonia, meningoencephalitis and/or arthritis (Taylor S. (2015). A review of equine sepsis. Equine veterinary education, 27(2), 99-109. https://doi.org/10.1111/eve.12290). Survivability of sepsis in neonates is grim with a survival rate between 45-60% (Taylor S. (2015). A review of equine sepsis. Equine veterinary education, 27(2), 99-109. https://doi.org/10.1111/eve.12290). Proinflammatory cytokines like TNFα and other cytokines down-stream of IL-23 were found to be present at high levels in sepsis foals and are directly linked to the development of multiple organ dysfunction syndrome (Taylor S. (2015). A review of equine sepsis. Equine veterinary education, 27(2), 99-109. https://doi.org/10.1111/eve.12290). To date, there arc no compounds for the treatment of sepsis and a compound targeting IL-23 and TNFα cytokines could be beneficial for the treatment of sepsis.

Anti-TNFα and anti-IL-23 monoclonal antibodies have been studied for the treatment of rheumatoid arthritis in humans (Smolen, J. S., Agarwal, S. K., Ilivanova, E., Xu, X. L., Miao, Y., Zhuang, Y., & Baker, D. (2017). A randomised phase II study evaluating the efficacy and safety of subcutaneously administered ustekinumab and guselkumab in patients with active rheumatoid arthritis despite treatment with methotrexate. Annals of the rheumatic diseases, 76(5), 831-839; Chimenti, M. S., Talamonti, M., Novelli, L., Teoli, M., Galluzzo, M., Triggianese, P., & Perricone, R. (2015). Long-term ustekinumab therapy of psoriasis in patients with coexisting rheumatoid arthritis and Sjögren syndrome. Report of two cases and review of literature. Journal of dermatological case reports, 9(3), 71-75. https://doi.org/10.3315/idcr.2015.1207; de Avila Machado, M. A., Maciel, A. A., de Lemos, L. L. P., Costa, J. O., Kakehasi, A. M., Andrade, E. I. G., . . . & de Assis Acurcio, F. (2013). Adalimumab in rheumatoid arthritis treatment: a systematic review and meta-analysis of randomized clinical trials. Revista Brasileira de Reumatologia (English Edition), 53(5), 419-430; Schiff, M. H., Burmester, G. R., Kent, J. D., Pangan, A. L., Kupper, H., Fitzpatrick, S. B., & Donovan, C. (2006). Safety analyses of adalimumab (HUMIRA) in global clinical trials and US postmarketing surveillance of patients with rheumatoid arthritis. Annals of the rheumatic diseases, 65(7), 889-894. https://doi.org/10.1136/ard.2005.043166). However, in recent studies of osteoarthritis in humans, both TNFα and IL-23 have been shown to be present in the synovial fluid of patients with OA (Scanzello, C. R., & Goldring, S. R. (2012). The role of synovitis in osteoarthritis pathogenesis. Bone, 51(2), 249-257. https://doi.org/10.1016/j.bone.2012.02.012; Askari, A., Naghizadeh, M. M., Homayounfar, R., Shahi, A., Afsarian, M. H., Paknahad, A., Kennedy, D., & Ataollahi, M. R. (2016). Increased Serum Levels of IL-17A and IL-23 Are Associated with Decreased Vitamin D3 and Increased Pain in Osteoarthritis. PloS one, 11(11), e0164757. https://doi.org/10.1371/journal.pgne.0164757; Scanzello, C. R., Umoh, E., Pessler, F., Diaz-Torne, C., Miles, T., Dicarlo, E., & Crow, M. K. (2009). Local cytokine profiles in knee osteoarthritis: elevated synovial fluid interleukin-15 differentiates early from end-stage disease. Osteoarthritis and cartilage, 17(8), 1040-1048). As osteoarthritis is characterized by inflammation in the joints, evidence of these pro-inflammatory cytokines in the synovial fluid is not surprising. Similar to humans, companion animals will often develop OA as they age. Like humans, OA results in pain, swollen joints, slow, stiffness or refusal to move as just a few symptoms (Brown, D. C. (2017). What can we learn from osteoarthritis pain in companion animals. Clin. Exp. Rheumatol, 35(Suppl 107), 53-58). Treatment for companion animals for OA includes anti-inflammatories; however, these can be hard on the kidneys of aging animals. Compounds targeting IL-23 and TNFα cytokines may be safer and more effective treatment for OA in companion animals.

There remains a need, therefore, for methods and compounds that can be used to bind companion animal IL23 in companion animals for treating IL23 associated conditions in companion animals. Ideally, such compounds would bind specifically to companion animal IL23 and have a half-life in plasma sufficiently long to be practicable for therapy but would not be highly immunogenic in companion animals.

### SUMMARY

The present disclosure relates generally to caninized, felinized, and equinized antibodies that specifically bind IL23 and/or TNFα, and the use of these antibodies in compositions and methods for treating inflammatory conditions in canines, felines, and equines, such as inflammatory bowel disease (IBD), osteoarthritis and gastroenteritis. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides caninized anti-IL23 antibodies, caninized anti-TNFα antibodies, felinized anti-IL23 antibodies, or felinized anti-TNFα antibodies. The present disclosure provides bispecific antibodies capable of binding both canine IL23 and canine TNFα (or both feline IL23 and feline TNFα), as well as methods for using such bispecific antibodies for methods of treatment in canines or felines, for example, treatment of IBD, sepsis, and gastroenteritis. The present disclosure provides methods and compositions for combination treatment for IBD in companion animals with anti-TNFα antibodies, and so relates to the fields of biology, molecular biology, and veterinary medicine.

Various embodiments provided by the present disclosure include, but are not limited to: Embodiment 1. An anti-IL23 antibody that binds to canine, feline, and/or equine IL23 comprising (i) a first light chain hypervariable region (HVR-L1), a second light chain hypervariable region (HVR-L2), and a third light chain hypervariable region (HVR-L3), and/or (ii) a first heavy chain hypervariable region (HVR-H1), a second heavy chain hypervariable region (HVR-H2), and a third heavy chain hypervariable region (HVR-H3), wherein: (a) the HVR-L1 region comprises amino acid sequence RASQGISSWLA (SEQ ID NO: 4), the HVR-L2 region comprises amino acid sequence YAASSLQS (SEQ ID NO: 5), and the HVR-L3 region comprises amino acid sequence QQYNIYPYT (SEQ ID NO: 6); and/or (b) the HVR-H1 region comprises amino acid sequence KGSGYSFITYWLG (SEQ ID NO: 8), the HVR-H2 region comprises amino acid sequence IMSPVDSDIR (SEQ ID NO: 9) and the HVR-H3 region comprises amino acid sequence ARRRPGQGYFDF (SEQ ID NO: 10).

Embodiment 2. The antibody of embodiment 1, wherein the antibody is caninized, felinized, or equinized.

Embodiment 3. The antibody of any one of embodiments 1-2, wherein the antibody comprises a light chain variable domain (VL) amino acid sequence having at least 90% identity to a sequence selected from SEQ ID NO: 3, 11, 13, and 15; and/or a heavy chain variable domain (VH) amino acid sequence having at least 90% identity to a sequence selected from SEQ ID NO: 7, 12, 14, and 16; optionally, wherein: (i) the light chain variable domain (VL) comprises a variant of SEQ ID NO: 3, 11, 13, and 15, wherein from 1 to 6 amino acids of the light chain variable domain (VL) are substituted by a different amino acid; and/or (ii) the heavy chain variable domain (VH) comprises a variant of SEQ ID NO: 7, 12, 14, and 16, wherein from 1 to 6 amino acids of the heavy chain variable domain (VH) are substituted by a different amino acid.

Embodiment 4. The antibody of any one of embodiments 1-3, wherein the antibody comprises a light chain variable domain (VL) comprises an amino acid sequence selected from SEQ ID NO: 3, 11, 13, and 15, and/or a heavy chain variable domain (VH) comprises an amino acid sequence selected from SEQ ID NO: 7, 12, 14, and 16; optionally, wherein: (i) the antibody comprises a light chain variable domain (VL) comprising an amino acid sequence selected from SEQ ID NO: 3 and a heavy chain variable domain (VH) comprising an amino acid sequence selected from SEQ ID NO: 7; (ii) the antibody comprises a light chain variable domain (VL) comprising an amino acid sequence selected from SEQ ID NO: 11 and a heavy chain variable domain (VH) comprising an amino acid sequence selected from SEQ ID NO: 12; (iii) the antibody comprises a light chain variable domain (VL) comprising an amino acid sequence selected from SEQ ID NO: 13 and a heavy chain variable domain (VH) comprising an amino acid sequence selected from SEQ ID NO: 14; or (iv) the antibody comprises a light chain variable domain (VL) comprising an amino acid sequence selected from SEQ ID NO: 15 and a heavy chain variable domain (VH) comprising an amino acid sequence selected from SEQ ID NO: 16.

Embodiment 5. The antibody of any one of embodiments 1-4, wherein the antibody comprises: a light chain (LC) amino acid sequence having at least 90% identity to an amino acid sequence selected from SEQ ID NO: 17, 19, 22, and 24, and/or a heavy chain (HC) amino acid sequence having at least 90% identity to SEQ ID NO: 18, 20, 21, 23, and 25; optionally, wherein the antibody comprises: (i) the LC amino acid sequence of SEQ ID NO: 17, and the HC amino acid sequence of SEQ ID NO: 18; (ii) the LC amino acid sequence of SEQ ID NO: 19, and the HC amino acid sequence of SEQ ID NO: 20; (iii) the LC amino acid sequence of SEQ ID NO: 19, and the HC amino acid sequence of SEQ ID NO: 21; (iv) the LC amino acid sequence of SEQ ID NO: 22, and the HC amino acid sequence of SEQ ID NO: 23; or (v) the LC amino acid sequence of SEQ ID NO: 24, and the HC amino acid sequence of SEQ ID NO: 25.

Embodiment 6. An anti-TNFα antibody that binds to canine, feline, and/or equine TNFα comprising (i) a first light chain hypervariable region (HVR-L1), a second light chain hypervariable region (HVR-L2), and a third light chain hypervariable region (HVR-L3), and/or (ii) a first heavy chain hypervariable region (HVR-H1), a second heavy chain hypervariable region (HVR-H2), and a third heavy chain hypervariable region (HVR-H3), wherein: (a) an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29); and/or (a) an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33).

Embodiment 7. The antibody of embodiment 6, wherein the antibody is caninized, felinized, or equinized.

Embodiment 8. The antibody of any one of embodiments 6-7, wherein the antibody comprises a light chain variable domain (VL) amino acid sequence having at least 90% identity to a sequence selected from SEQ ID NO: 26, 34, and 36; and/or a heavy chain variable domain (VH) amino acid sequence having at least 90% identity to a sequence selected from SEQ ID NO: 30, 35, and 37; optionally, wherein: (i) the light chain variable domain (VL) comprises a variant of SEQ ID NO: 26, 34, and 36, wherein from 1 to 6 amino acids of the light chain variable domain (VL) are substituted by a different amino acid; and/or (ii) the heavy chain variable domain (VH) comprises a variant of SEQ ID NO: 30, 35, and 37, wherein from 1 to 6 amino acids of the heavy chain variable domain (VH) are substituted by a different amino acid.

Embodiment 9. The antibody of any one of embodiments 6-8, wherein the antibody comprises a light chain variable domain (VL) comprises an amino acid sequence selected from SEQ ID NO: 26, 34, and 36, and/or a heavy chain variable domain (VH) comprises an amino acid sequence selected from SEQ ID NO: 30, 35, and 37; optionally, wherein the antibody comprises: (i) a light chain variable domain (VL) comprising an amino acid sequence selected from SEQ ID NO: 26 and a heavy chain variable domain (VH) comprising an amino acid sequence selected from SEQ ID NO: 30; (ii) a light chain variable domain (VL) comprising an amino acid sequence selected from SEQ ID NO: 34 and a heavy chain variable domain (VH) comprising an amino acid sequence selected from SEQ ID NO: 35; (iii) a light chain variable domain (VL) comprising an amino acid sequence selected from SEQ ID NO: 36 and a heavy chain variable domain (VH) comprising an amino acid sequence selected from SEQ ID NO: 37; (iv) a light chain variable domain (VL) comprising an amino acid sequence selected from SEQ ID NO: 36 and a heavy chain variable domain (VH) comprising an amino acid sequence selected from SEQ ID NO: 37; (v) a light chain variable domain (VL) comprising an amino acid sequence selected from SEQ ID NO: 36 and a heavy chain variable domain (VH) comprising an amino acid sequence selected from SEQ ID NO: 30; or (vi) a light chain variable domain (VL) comprising an amino acid sequence selected from SEQ ID NO: 26 and a heavy chain variable domain (VH) comprising an amino acid sequence selected from SEQ ID NO: 37.

Embodiment 10. The antibody of any one of embodiments 6-9, wherein the antibody comprises: a light chain (LC) amino acid sequence having at least 90% identity to a sequence selected from SEQ ID NO: 38, 40, and 41 and/or a heavy chain (HC) amino acid sequence having at least 90% identity to a sequence selected from SEQ ID NO: 39, 42, 43, and 44; optionally, wherein the antibody comprises: (i) the LC amino acid sequence of SEQ ID NO: 38, and the HC amino acid sequence of SEQ ID NO: 39; (ii) the LC amino acid sequence of SEQ ID NO: 40, and the HC amino acid sequence of SEQ ID NO: 42; (iii) the LC amino acid sequence of SEQ ID NO: 41, and the HC amino acid sequence of SEQ ID NO: 42; (iv) the LC amino acid sequence of SEQ ID NO: 40, and the HC amino acid sequence of SEQ ID NO: 43; (v) the LC amino acid sequence of SEQ ID NO: 41, and the HC amino acid sequence of SEQ ID NO: 43; (vi) the LC amino acid sequence of SEQ ID NO: 40, and the HC amino acid sequence of SEQ ID NO: 44; or (vi) the LC amino acid sequence of SEQ ID NO: 41, and the HC amino acid sequence of SEQ ID NO: 44.

Embodiment 11. The antibody of any one of embodiments 6-10, wherein the antibody is an scFv antibody; optionally, wherein the scFv antibody comprises an amino acid sequence of at least 90% sequence identity to a sequence selected from SEQ ID NO: 45 and 46.

Embodiment 12. The antibody of any one of embodiments 1-11, wherein the antibody is an antibody fragment selected from Fv, scFv, Fab, Fab′, F(ab′)2, and Fab′-SH.

Embodiment 13. The antibody of any one of embodiments 1-12, wherein the antibody comprises a canine heavy chain constant region selected from an IgG-A, IgG-B, IgG-C, and IgG-D constant region.

Embodiment 14. The antibody of any one of embodiments 1-13, wherein the antibody comprises: (i) a canine light chain constant region and/or a canine heavy chain constant region; (ii) a feline light chain constant region and/or a feline heavy chain constant region; or (iii) an equine light chain constant region and/or an equine heavy chain constant region.

Embodiment 15. The antibody of any one of embodiments 1-14, wherein the antibody comprises a heavy chain constant region with a “Y” mutation at EU Numbering position 252.

Embodiment 16. A bispecific antibody that binds to canine IL23 and canine TNFα, wherein the antibody comprises: a light chain (LC) comprising a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6); a heavy chain (HC) comprising a VH domain having an HVR-H1 region comprising an amino acid sequence KGSGYSFITYWLG (SEQ ID NO: 8), an HVR-H2 region comprising an amino acid sequence IMSPVDSDIR (SEQ ID NO: 9), and an HVR-H3 region comprising an amino acid sequence ARRRPGQGYFDF (SEQ ID NO: 10); and an scFv antibody fused to the HC, wherein the scFv antibody comprises a VL domain having an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29); and a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33).

Embodiment 17. The bispecific antibody of embodiment 16, wherein the antibody comprises: a light chain (LC) comprising a light chain variable domain (VL) amino acid sequence having at least 90% identity to a sequence selected from SEQ ID NO: 3, 11, 13, and 15, and a heavy chain (HC) comprising a heavy chain variable domain (VH) amino acid sequence having at least 90% identity to a sequence selected from SEQ ID NO: 7, 12, 14, and 16, wherein the HC is fused to an scFv antibody comprising a light chain variable domain (VL) amino acid sequence having at least 90% identity to a sequence selected from SEQ ID NO: 26, 34, and 36 and a heavy chain variable domain (VH) amino acid sequence having at least 90% identity to a sequence selected from SEQ ID NO: 30, 35, and 37.

Embodiment 18. The bispecific antibody of any one of embodiments 16-17, wherein the light chain (LC) comprises an amino acid sequence having at least 90% identity to a sequence selected from SEQ ID NO: 19 and 22; and the heavy chain (HC) comprises an amino acid sequence having at least 90% sequence identity to SEQ ID NO: 47 and 48; optionally, wherein the antibody comprises: (i) the LC amino acid sequence of SEQ ID NO: 19, and the HC amino acid sequence of SEQ ID NO: 47; (ii) the LC amino acid sequence of SEQ ID NO: 19, and the HC amino acid sequence of SEQ ID NO: 48; (iii) the LC amino acid sequence of SEQ ID NO: 22, and the HC amino acid sequence of SEQ ID NO: 47; or (iv) the LC amino acid sequence of SEQ ID NO: 22, and the HC amino acid sequence of SEQ ID NO: 48.

Embodiment 19. A bispecific antibody that binds to canine IL23 and canine TNFα, wherein the antibody comprises: a light chain (LC) comprising a VL domain an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29), and a heavy chain (HC) comprising a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33), and an scFv antibody fused to the HC, wherein the scFv antibody comprises a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6), and a VH domain having an HVR-H1 region comprising an amino acid sequence KGSGYSFITYWLG (SEQ ID NO: 8), an HVR-H2 region comprising an amino acid sequence IMSPVDSDIR (SEQ ID NO: 9) and an HVR-H3 region comprising an amino acid sequence ARRRPGQGYFDF (SEQ ID NO: 10).

Embodiment 20. The bispecific antibody of embodiment 18, wherein the antibody comprises: a light chain (LC) comprising a light chain variable domain (VL) amino acid sequence having at least 90% identity to a sequence selected from SEQ ID NO: 26, 34, and 36, and a heavy chain variable domain (VH) amino acid sequence having at least 90% identity to a sequence selected from SEQ ID NO: 30, 35, and 37, wherein the HC is fused to an scFv antibody comprising a light chain variable domain (VL) amino acid sequence having at least 90% identity to a sequence selected from SEQ ID NO: 3, 11, 13, and 15 and a heavy chain variable domain (VH) amino acid sequence having at least 90% identity to a sequence selected from SEQ ID NO: 7, 12, 14, and 16.

Embodiment 21. A bispecific antibody that binds to canine, feline, and/or equine IL23 and canine, feline, and/or equine TNFα, wherein the antibody comprises:
(i) an anti-IL23 light chain (LC) comprising a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6);
(ii) an anti-IL23 heavy chain (HC) comprising a VH domain having an HVR-H1 region comprising an amino acid sequence KGSGYSFITYWLG (SEQ ID NO: 8), an HVR-H2 region comprising an amino acid sequence IMSPVDSDIR (SEQ ID NO: 9), and an HVR-H3 region comprising an amino acid sequence ARRRPGQGYFDF (SEQ ID NO: 10);
(iii) an anti-TNFα light chain (LC) comprising a VL domain an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29); and
(iv) an anti-TNFα heavy chain (HC) comprising a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33).

Embodiment 22. The bispecific antibody of embodiment 21, wherein:
(i) the anti-IL23 light chain (LC) comprises a light chain variable domain (VL) amino acid sequence having at least 90% identity to a sequence selected from SEQ ID NO: 3, 11, 13, and 15;
(ii) the anti-IL23 heavy chain (HC) comprises a heavy chain variable domain (VH) amino acid sequence having at least 90% identity to a sequence selected from SEQ ID NO: 7, 12, 14, and 16;
(iii) the anti-TNFα light chain (LC) comprises a light chain variable domain (VL) amino acid sequence having at least 90% identity to a sequence selected from SEQ ID NO: 26, 34, and 36; and
(iv) the anti-TNFα heavy chain (HC) comprises a heavy chain variable domain (VH) amino acid sequence having at least 90% identity to a sequence selected from SEQ ID NO: 30, 35, and 37.

Embodiment 23. The bispecific antibody of any one of embodiments 21-22, wherein:
(i) the anti-IL23 light chain (LC) comprises an amino acid sequence having at least 90% identity to a sequence selected from SEQ ID NO: 19 and 22;
(ii) the anti-IL23 heavy chain (HC) comprises an amino acid sequence having at least 90% sequence identity to SEQ ID NO: 20, 21, and 23;
(iii) the anti-TNFα light chain (LC) comprises an amino acid sequence having at least 90% identity to a sequence selected from SEQ ID NO: 40 and 41; and
(iv) the anti-TNFα heavy chain (HC) comprises an amino acid sequence having at least 90% sequence identity to SEQ ID NO: 42, 43, and 44.

Embodiment 24. An isolated nucleic acid or nucleic acids encoding the antibody of any one of embodiments 1 to 23.

Embodiment 25. A host cell comprising the nucleic acid or nucleic acids of embodiment 24.

Embodiment 26. A method of producing an antibody comprising culturing the host cell of embodiment 25 and isolating the antibody.

Embodiment 27. A pharmaceutical composition comprising the antibody of any one of embodiments 1 to 23 and a pharmaceutically acceptable carrier.

Embodiment 28. A method of treating a canine, feline, or equine having a condition associated with IL23, the method comprising administering to the canine, feline, or equine a therapeutically effective amount of the antibody of any one of embodiments 1 to 23 or the pharmaceutical composition of embodiment 27.

Embodiment 29. A method of maintaining remission of a condition associated with IL23 in a canine, feline, or equine, the method comprising administering to the canine, feline, or equine a therapeutically effective amount of the antibody of any one of embodiments 1 to 23 or the pharmaceutical composition of embodiment 27.

Embodiment 30. The method of any one of embodiments 28-29, wherein the condition associated with IL23 is an inflammatory disease.

Embodiment 31. The method of any one of embodiments 28-30, wherein the condition associated with IL23 is a gastrointestinal inflammatory disease.

Embodiment 32. The method of any one of embodiments 28-31, wherein the condition associated with IL23 is inflammatory bowel disease.

Embodiment 33. The method of any one of embodiments 28-32, wherein the condition associated with IL23 is ankylosing spondylitis, asthma, cancer, Crohn's disease, idiopathic arthritis, psoriasis, plaque psoriasis, psoriatic arthritis, rheumatoid arthritis, osteoarthritis or ulcerative colitis.

Embodiment 34. A method of treating a canine, feline, or equine having a condition associated with IL23 and TNFα, the method comprising administering to the canine, feline, or equine a therapeutically effective amount of an IL23 antibody embodiments 1-5 or 12-23, and anti-TNFα antibody of any one of embodiments 6-23, or a pharmaceutical composition of embodiment 24.

Embodiment 35. The method of embodiment 34, wherein the anti-IL23 antibody is administered in combination with an anti-TNFα antibody or in the form of a bispecific IL23/TNFα antibody, wherein the condition associated with IL23 or IL23/TNFα is a gastrointestinal inflammatory disease.

Embodiment 36. The method of any one of embodiments 34 to 35, wherein the condition associated with IL23 or IL23/TNFα is ankylosing spondylitis, asthma, cancer, Crohn's disease, idiopathic arthritis, psoriasis, plaque psoriasis, psoriatic arthritis, rheumatoid arthritis, osteoarthritis or ulcerative colitis.

Embodiment 37. The method of any one of embodiments 28 to 36, wherein the antibody or the pharmaceutical composition is administered parenterally.

Embodiment 38. The method of any one of embodiments 28 to 37, wherein the antibody or the pharmaceutical composition is administered by an intramuscular route, an intraperitoneal route, an intracerebrospinal route, a subcutaneous route, an intra-arterial route, an intrasynovial route, an intrathecal route, or an inhalation route.

Embodiment 39. The method of any one of embodiments 28 to 38, wherein the method further comprises administering an IL17 antibody, an IL-5 antibody, an IL-31 antibody, an IL4 antibody, an IL13 antibody, an IL23 antibody, an IgE antibody, a CD11α antibody, an IL6R antibody, an α4-intergrin antibody, an beta9-integrin, an IL12 antibody, an IL10 antibody, or an anti-Bly S antibody.

Embodiment 40. The method of any one of embodiments 28 to 39, wherein the antibody is administered at an amount in the range of 0.01 mg/kg body weight to 100 mg/kg body weight per dose.

Embodiment 41. A method of reducing canine, feline, or equine IL23 and/or TNFα signaling function in a cell, the method comprising exposing the cell to an antibody of any one of embodiments 1 to 23 under conditions permissive for binding of the antibody to IL23 and/or TNFα, thereby reducing binding to IL23 and/or TNFα signaling function by the cell.

Embodiment 42. The method of embodiment 41, wherein the cell is exposed to the antibody or the pharmaceutical composition ex vivo.

Embodiment 43. The method of embodiment 41, wherein the cell is exposed to the antibody or the pharmaceutical composition in vivo.

Embodiment 44. The method of any one of embodiments 41-43, wherein the cell is a canine, feline, or equine cell.

Embodiment 45. A method for detecting IL23 and/or TNFα in a sample from a companion animal species comprising contacting the sample with the antibody of any one of embodiments 1 to 23 under conditions permissive for binding of the antibody to IL23 and/or TNFα, and detecting whether a complex is formed between the antibody and IL23 and/or TNFα in the sample.

Embodiment 46. The method of embodiment 45, wherein the sample is a biological sample obtained from a canine, a feline or an equine.

### DETAILED DESCRIPTION OF THE EMBODIMENTS

The present disclosure provides a detailed description, including examples, of antibodies that specifically bind to canine IL23 and/or feline IL23, antibodies that specifically bind to canine TNFα and/or feline TNFα, as well as bispecific antibodies that bind to both canine IL23 and canine TNFα (or feline IL23 and feline TNF). The present disclosure provides various exemplary forms of these antibodies, including full-length antibodies and scFv antibodies, and uses of these antibodies, including method of treatment of various diseases and disorders that are mediated or associated with the binding activity of IL23 and/or TNFα. Methods of designing, producing, or purifying bispecific antibodies to canine IL23 and canine TNFα are also provided. Methods of detecting IL23 and/or TNFα in a sample from a companion animal species are provided.

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “i to 50,” includes “2 to 25,” “5 to 20,” “25 to 50,” “i to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology. Vols. 00-130 (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

“IL23” or “IL-23” as used herein, refers to the cytokine protein interleukin 23 (or IL-23), and encompasses the IL23 proteins from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of IL23, e.g., splice variants or allelic variants. Amino acid sequences of exemplary recombinant forms of canine IL23 and feline IL23 proteins are provided in Table 1 below and the attached Sequence Listing.

“TNF” or “TNFα” or “TNFα” as used herein refers to the cytokine protein, tumor necrosis factor, the TNFα proteins from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of TNFα, e.g., splice variants or allelic variants.

“IL23 mediated condition” or “IL23 mediated disease,” as used herein, encompasses any disease or disorder associated with the specific binding of IL23 to the IL23 receptor or other specific IL23 binding targets, and can include a disease associated with, caused by, or characterized by, elevated levels or altered gradients of IL23 concentration. For example, specific binding of IL23 stimulates production of Th17 cells involved in an immune response. Accordingly, IL23 mediated diseases can include, but are not limited to, any disease or condition mediated by and/or responsive to antagonists or inhibitors of IL23 binding to IL23 receptor or other IL23 targets. Specific exemplary diseases or conditions include, but are not limited to Crohn's disease, inflammatory bowel disease (IBD), psoriasis, including plaque psoriasis, psoriatic arthritis, rheumatoid arthritis, ulcerative colitis, osteo arthritis, multiple sclerosis and other chronic inflammatory disorders.

“TNFα mediated condition” or “TNFα mediated disease,” as used herein, encompasses any disease or disorder associated with the specific binding of TNFα to a TNFα receptor or other specific TNFα binding target, and can include a disease associated with, caused by, or characterized by, elevated levels or altered gradients of TNFα concentration. Accordingly, TNFα mediated diseases can include, but are not limited to, any disease or condition mediated by and/or responsive to antagonists or inhibitors of TNFα binding to TNFα receptor or other specific TNFα binding targets. Specific exemplary diseases are provided elsewhere herein.

“Antibody,” as used herein, refers to a molecule comprising one or more polypeptide chains that specifically binds to, or is immunologically reactive with, a particular antigen. Exemplary antibodies of the present disclosure include monoclonal antibodies, polyclonal antibodies, chimeric antibodies, caninized antibodies, felinized antibodies, multispecific (or heteroconjugate) antibodies (e.g., bispecific antibodies), monovalent antibodies (e.g., single-arm antibodies), multivalent antibodies, antigen-binding fragments (e.g., Fab′, F(ab′)₂, Fab, Fv, rIgG, and scFv fragments), antibody fusions, and synthetic antibodies (or antibody mimetics).

“Anti-IL23 antibody” or “antibody that binds IL23” refers to an antibody that binds IL23 with sufficient affinity such that the antibody is useful as a diagnostic and/or therapeutic agent in targeting IL23. Anti-IL23 antibody may be further specified to refer to an antibody that binds to a specific type of IL23, such as canine IL23 (e.g., “anti-canine-IL23” or “anti-IL23 that binds canine IL23”). In some embodiments, the extent of binding of an anti-IL23 specific antibody to an unrelated, non-IL23 antigen is less than about 20%, less than about 15%, less than about 10%, or less than about 5% of the binding of the antibody to IL23 as measured, e.g., by a radioimmunoassay (RIA) or surface plasmon resonance (SPR). In some embodiments, an antibody that binds to IL23 has a dissociation constant (K_{D}) of <1 μM, <100 nM, <10 nM, <1 nM, <0.1 nM, <0.01 nM, or <1 μM (e.g., 10⁻⁸ M or less, e.g., from 10⁻⁸ M to 10⁻¹³ M, e.g., from 10⁻⁹ M to 10⁻¹³ M).

“Anti-TNFα antibody” or “antibody that binds TNF” refers to an antibody that binds TNFα with sufficient affinity such that the antibody is useful as a diagnostic and/or therapeutic agent in targeting TNFα. Anti-TNFα antibody may be further specified to refer to an antibody that binds to a specific type of TNFα, such as canine TNFα (e.g., “anti-canine-TNFα” or “anti-TNFα that binds canine TNFα”). In some embodiments, the extent of binding of an anti-TNFα specific antibody to an unrelated, non-TNFα antigen is less than about 20%, less than about 15%, less than about 10%, or less than about 5% of the binding of the antibody to TNFα as measured, e.g., by a radioimmunoassay (RIA) or surface plasmon resonance (SPR). In some embodiments, an antibody that binds to TNFα has a dissociation constant (K_{D}) of <1 μM, <100 nM, <10 nM, <1 nM, <0.1 nM, <0.01 nM, or <1 μM (e.g., 10⁻⁸ M or less, e.g., from 10⁻⁸ M to 10⁻¹³ M, e.g., from 10⁻⁹ M to 10⁻¹³ M).

“Full-length antibody,” “intact antibody,” or “whole antibody” are used herein interchangeably to refer to an antibody having a structure substantially similar to a native antibody structure or having heavy chains that contain an Fc region as defined herein.

“Class” of an antibody refers to the type of constant domain or constant region possessed by its heavy chain. There are five major classes of human antibodies: IgA, IgD, IgE, IgG, and IgM, and several of these are further divided into subclasses (isotypes), e.g., IgG1, IgG2, IgG3, IgG4, IgAQ1, and IgA2. The heavy chain constant domains that correspond to the different classes of immunoglobulins are called α, δ, ε, γ, and μ, respectively. Canine, feline, and equine species have different classes of antibodies that are shared by many other mammalian species. For example, canine species have antibody classes, IgGA, IgGB, IgGC, IgGD, and feline species have antibody classes, IgGAl, IgGA2, IgGB.

“Variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three hypervariable regions (HVRs) (see, e.g., Kindt et al., Kuby Immunology, 6^{th} ed., W.H. Freeman and Co., page 91). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (see, e.g., Portolano et al., J. Immunol. 150:880-887 (1993); Clarkson et al., Nature 352:624-628 (1991)).

“Hypervariable region” or “HVR,” as used herein, refers to each of the regions of an antibody variable domain which are hypervariable in sequence and/or form structurally defined loops (“hypervariable loops”). Generally, native antibodies comprise four chains with six HVRs; three in the heavy chain variable domain, V_{H} (HVR-H1, HVR-H2, HVR-H3), and three in the light chain variable domain, V_{L} (HVR-L1, HVR-L2, HVR-L3). The HVRs generally comprise amino acid residues from the hypervariable loops and/or from the “complementarity determining regions” (CDRs). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops and are used by Oxford Molecular's AbM antibody modeling software. The “contact” hypervariable regions are based on an analysis of the available complex crystal structures. The residues from each of these hypervariable regions are noted in the table below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B¹ | H26-H35B¹ | H26-H32¹ | H30-H35B¹ |
| | H31-H35² | H26-H35² | H26-H32² | H30-H35² |
| H2 | H50-H65 | H50-H58 | H53-H55 | H47-H58 |
| H3 | H95-H102 | H95-H102 | H96-H101 | H93-H101 |

¹Kabat numbering
²Chothia numbering

Unless otherwise indicated, HVR residues and other residues in the variable domain (e.g., FR residues) are numbered herein according to Kabat et al., supra.

Hypervariable regions, as used herein, may include extended or alternative hypervariable regions as follows: 24-36 or 24-34 (L1), 46-56 or 50-56 (L2) and 89-97 or 89-96 (L3) in the V_{L} domain and 26-35 or 30-35 (H1), 50-61, 50-65 or 49-65 (H2) and 93-102, 94-102, or 95-102 (H3) in the V_{H} domain. The variable domain residues are numbered according to Kabat et al., supra, for each of these definitions.

“Complementarity determining region,” or “CDR,” as used herein, refers to the regions within the HVRs of the variable domain which have the highest sequence variability and/or are involved in antigen recognition. Generally, native antibodies comprise four chains with six CDRs; three in the heavy chain variable domains, V_{H} (H1, H2, H3), and three in the light chain variable domains, V_{L} (L1, L2, L3). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35 of H1, 50-61 of H2, and 95-102 of H3. (Numbering according to Kabat et al., supra).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in V_{H} (or V_{L}): FRI-H1(L1)-FR2-H2(L2)-FR3-H3(L3)-FR4.

“Native antibody” refers to a naturally occurring immunoglobulin molecule. For example, native IgG antibodies are heterotetrameric glycoproteins of about 150,000 Daltons, composed of two identical light chains and two identical heavy chains that are disulfide-bonded. From N- to C-terminus, each heavy chain has a variable region (V_{H}), also called a variable heavy domain or a heavy chain variable domain, followed by three constant domains (CH1, CH2, and CH3). Similarly, from N- to C-terminus, each light chain has a variable region (V_{L}), also called a variable light domain or a light chain variable domain, followed by a constant light (CL) domain. The light chain of an antibody may be assigned to one of two types, called kappa (u) and lambda (k), based on the amino acid sequence of its constant domain.

“Monoclonal antibody” as used herein refers to an antibody obtained from a substantially homogeneous population of antibodies, i.e., the individual antibodies comprising the population are identical and/or bind the same epitope, except for possible variant antibodies (e.g., variant antibodies contain mutations that occur naturally or arise during production of a monoclonal antibody, and generally are present in minor amounts). In contrast to polyclonal antibody preparations, which typically include different antibodies directed against different determinants (epitopes), each monoclonal antibody of a monoclonal antibody preparation is directed against a single determinant on an antigen. Thus, the term “monoclonal” indicates the character of the antibody as being obtained from a substantially homogeneous population of antibodies and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Chimeric antibody” refers to an antibody in which a portion of the heavy and/or light chain is derived from a particular source or species, while the remainder of the heavy and/or light chain is derived from a different source or species. A “canine chimeric antibody” refers to a chimeric antibody having at least a portion of a heavy chain or a portion of a light chain are derived from a dog. In some embodiments, a canine chimeric antibody can include mouse VH and/or VL sequences and canine heavy and light chain constant domains. I n some embodiments, the antibody is a chimeric antibody comprising murine heavy chain variable domain (VH) framework regions or murine light chain variable domain (VL) framework regions.

“Caninized antibody” refers to a chimeric antibody comprising amino acid sequences from non-canine HVRs and amino acid sequences from canine FRs. In certain embodiments, a caninized antibody will comprise substantially all of at least one, and typically two, variable domains, in which all or substantially all of the HVRs correspond to those of a non-canine antibody, and all or substantially all of the FRs correspond to those of a canine antibody. A caninized antibody optionally may comprise at least a portion of an antibody constant region derived from a canine antibody. A “caninized form” of an antibody, e.g., a non-canine antibody, refers to an antibody that has undergone caninization.

“Felinized antibody” refers to a chimeric antibody comprising amino acid sequences from non-feline HVRs and amino acid sequences from feline FRs. In certain embodiments, a felinized antibody will comprise substantially all of at least one, and typically two, variable domains, in which all or substantially all of the HVRs correspond to those of a non-feline antibody, and all or substantially all of the FRs correspond to those of a feline antibody. A felinized antibody optionally may comprise at least a portion of an antibody constant region derived from a feline antibody. A “felinized form” of an antibody, e.g., a non-feline antibody, refers to an antibody that has undergone felinization.

“Canine antibody” refers to an antibody which possesses an amino acid sequence corresponding to that of an antibody produced by a canine or a canine cell or derived from a non-canine source that utilizes canine antibody repertoires or other canine antibody-encoding sequences. This definition of a canine antibody specifically excludes a caninized antibody comprising non-canine antigen-binding residues.

“Consensus framework” is a framework which represents the most commonly occurring amino acid residues in a selection of immunoglobulin V_{L} or V_{H} framework sequences. Generally, the selection of immunoglobulin V_{L} or V_{H} sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the V_{L}, the subgroup is subgroup kappa I as in Kabat et al., supra. In some embodiments, for the V_{H}, the subgroup is subgroup III as in Kabat et al., supra.

“Acceptor framework” as used herein is a framework comprising the amino acid sequence of a light chain variable domain (V_{L}) framework or a heavy chain variable domain (V_{H}) framework derived from an immunoglobulin framework or a consensus framework. An acceptor framework “derived from” an immunoglobulin framework or a consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor framework is identical in sequence to the V_{L} immunoglobulin framework sequence or consensus framework sequence.

“Fc region,” refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. The Fc sequence of an immunoglobulin generally comprises two constant domains, a CH2 domain and a CH3 domain, and optionally comprises a CH4 domain. The boundaries of the Fc sequence of an immunoglobulin heavy chain may vary depending on immunoglobulin class and species.

The term “IgX Fc” means the Fc region is derived from a particular antibody isotype (e.g., IgG, IgA, IgD, IgE, IgM, etc.), where “X” denotes the antibody isotype. Thus, “IgG Fc” denotes the Fc region of a γ chain, “IgA Fc” denotes the Fc region of an α chain, “IgD Fc” denotes the Fc region of a δ chain, “IgE Fc” denotes the Fc region of an ε chain, “IgM Fc” denotes the Fc region of a μ chain, etc. In some embodiments, the IgG Fc region comprises CH1, hinge, CH2, CH3, and CL1. “IgX-N-Fc” denotes that the Fc region is derived from a particular subclass of antibody isotype (such as canine IgG subclass A, B, C, or D; or feline IgG subclass 1, 2a, or 2b), where “N” denotes the subclass. In some embodiments, IgX Fc or IgX-N-Fc regions are derived from a companion animal, such as a dog. In some embodiments, IgG Fc regions are isolated from canine γ heavy chains, such as IgG-A, IgG-B, IgG-C, or IgG-D. Antibodies comprising an Fc region of IgG-A, IgG-B, IgG-C, or IgG-D may provide for higher expression levels in recombination production systems. “IgX Fc” and “IgX Fc polypeptide” are intended to include wild-type IgX Fc polypeptides and variant IgX Fc polypeptides.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

“Immunoconjugate” refers to an antibody conjugated to one or more heterologous molecule(s), including but not limited to a cytotoxic agent.

“Multispecific antibody” is an antibody having at least two different binding sites, each site with a different binding specificity. A multispecific antibody can be a full-length antibody or an antibody fragment, and the different binding sites may bind each to a different antigen or the different binding sites may bind to two different epitopes of the same antigen.

“Fv fragment” refers to an antibody fragment which contains a complete antigen recognition and binding site. This region consists of a dimer of one heavy and one light chain variable domain in tight association, which can be covalent in nature, for example in scFv. It is in this configuration that the three HVRs of each variable domain interact to define an antigen binding site on the surface of the V_{H}-V_{L} dimer. Collectively, the six HVRs or a subset thereof confer antigen binding specificity to the antibody. However, even a single variable domain (or half of an Fv comprising only three HVRs specific for an antigen) has the ability to recognize and bind antigen, although usually at a lower affinity than the entire binding site.

“Fab fragment’ refers to an antibody fragment that contains a variable and constant domain of the light chain and a variable domain and the first constant domain (CH1) of the heavy chain. “F(ab′)₂ fragments” comprise a pair of Fab fragments which are generally covalently linked near their carboxy termini by hinge cysteines between them. Other chemical couplings of antibody fragments also are known in the art.

“Antigen binding arm,” as used herein, refers to a component of an antibody that has an ability to specifically bind a target molecule of interest. Typically the antigen binding arm is a complex of immunoglobulin polypeptide sequences, e.g., HVR and/or variable domain sequences of an immunoglobulin light and heavy chain.

“Single-chain Fv” or “scFv” refer to antibody fragments comprising the V_{H} and V_{L} domains of an antibody, wherein these domains are present in a single polypeptide chain. Generally, an scFv antibody comprises a single-chain polypeptide with a polypeptide linker between the polypeptides comprising the V_{H} domain and the V_{L} domain sequences. The V_{H} and V_{L} domains fused by a polypeptide linker allows the hypervariable regions of the scFv to form the desired antigen binding structure.

“Polypeptide linker” or “polypeptide linker” as used herein refers to a chain of two or more amino acids with each end of the chain covalently attached to a different polypeptide molecule, thereby functioning to conjugate or fuse the different polypeptides.

“Diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites.

“Linear antibodies” refers to the antibodies described in Zapata et al., Protein Eng., 8(10): 1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

“Naked antibody” refers to an antibody that is not conjugated to a heterologous moiety (e.g., a cytotoxic moiety) or radiolabel.

“Affinity” refers to the strength of the sum total of noncovalent interactions between a single binding site of a molecule (e.g., an antibody) and its binding partner (e.g., an antigen). “Binding affinity” refers to intrinsic binding affinity which reflects a 1:1 interaction between members of a binding pair (e.g., antibody and antigen). The affinity of a molecule X for its partner Y can generally be represented by the equilibrium dissociation constant (K_{D}). Affinity can be measured by common methods known in the art, including those described herein. Specific illustrative and exemplary embodiments for measuring binding affinity are described in the following.

“Binds specifically” or “specific binding” refers to binding of an antibody to an antigen with an affinity value of no more than about 1×10⁻⁷ M. In some embodiments, an antibody may have a secondary affinity for an antigen other than the antigen to which it binds specifically, where “secondary affinity” will generally refer to binding of an antibody to a secondary antigen with an affinity value of more than about 10 nM as described elsewhere herein. Where an antibody may have a secondary affinity for a secondary antigen, such an antibody will nevertheless bind specifically to the primary antigen.

“Isolated antibody” refers to an antibody which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic methods (e.g., ion exchange or reverse phase HPLC). For review of methods for assessment of antibody purity, see, e.g., Flatman et al., J. Chromatogr. B 848:79-87.

As used herein, “percent (%) amino acid sequence identity” and “homology” with respect to a polypeptide, or antibody sequence are defined as the percentage of amino acid residues in a candidate sequence that are identical with the amino acid residues in the specific peptide or polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

An “amino acid substitution” refers to the replacement of one amino acid in a polypeptide with another amino acid. In some embodiments, an amino acid substitution is a conservative substitution. Amino acid substitutions may be introduced into a molecule of interest and the products screened for a desired activity, for example, retained/improved antigen binding, decreased immunogenicity, or improved ADCC or CDC or enhanced pharmacokinetics.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters or enhancers) that regulate the expression of the polypeptide of interest, or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NS0 cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “companion animal species” refers to an animal suitable to be a companion to humans. In some embodiments, a companion animal species is a small mammal, such as a canine, feline, dog, cat, horse, rabbit, ferret, guinea pig, rodent, etc. In some embodiments, a companion animal species is a large animal like camel or farm animal, such as a horse, cow, pig, etc.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

“Substantially similar” or “substantially the same,” as used herein, refers to a sufficiently high degree of similarity between two numeric values (for example, one associated with a test antibody and the other associated with a reference antibody), such that one of skill in the art would consider the difference between the two values to be of little or no biological and/or statistical significance within the context of the biological characteristic measured by said values (e.g., K_{D} values).

“Substantially different,” as used herein, refers to a sufficiently high degree of difference between two numeric values (generally one associated with a molecule and the other associated with a reference molecule) such that one of skill in the art would consider the difference between the two values to be of statistical significance within the context of the biological characteristic measured by said values (e.g., K_{D} values).

“Treatment,” “treat” or “treating” refers to intervention in an attempt to alter the natural course of a disorder in the individual being treated and can be performed either for prophylaxis or during the course of clinical pathology. Desired results of treatment can include, but are not limited to, preventing occurrence or recurrence of the disorder, alleviation of symptoms, diminishment of any direct or indirect pathological consequences of the disorder, preventing metastasis, decreasing the rate of progression, amelioration or palliation of a disease state, and remission or improved prognosis. For example, treatment can include administration of a therapeutically effective amount of pharmaceutical formulation comprising an anti-IL23 antibody to a subject to delay development or slow progression of a disease or condition mediated by IL23 or disease or condition in which IL23 may play a role in the pathogenesis and/or progression. Treatment does not require one-hundred percent removal of all aspects of the disorder.

“Pharmaceutical formulation” refers to a preparation in a form that allows the biological activity of the active ingredient(s) to be effective, and which contain no additional components which are toxic to the subjects to which the formulation is administered. A pharmaceutical formulation may include one or more active agents. For example, a pharmaceutical formulation may include an anti-IL23 antibody as the sole active agent of the formulation or may include an anti-IL23 antibody and one or more additional active agents.

“Pharmaceutically acceptable carrier” refers to an ingredient in a pharmaceutical formulation, other than an active ingredient, which is nontoxic to the subject to whom it is administered. A pharmaceutically acceptable carrier includes, but is not limited to, a buffer, excipient, stabilizer, or preservative.

“Therapeutically effective amount” refers to the amount of an active ingredient or agent (e.g., a pharmaceutical formulation) to achieve a desired therapeutic or prophylactic result, e.g., to treat or prevent a disease, disorder, or condition in a subject. In the case of a IL23 mediated disease or condition, the therapeutically effective amount of the therapeutic agent is an amount that reduces, prevents, inhibits, and/or relieves to some extent one or more of the symptoms associated with the disease, disorder, or condition.

“Individual” or “subject” refers to a mammal, including but not limited to, domesticated or companion animals (e.g., cows, sheep, cats, dogs, and horses), primates (e.g., humans and non-human primates such as monkeys), rabbits, and rodents (e.g., mice and rats).

### Anti-IL23 and Anti-TNFα Antibodies

Table 1 below provides a summary description of the sequences referenced in the present disclosure, including canine IL23 protein, feline IL23 protein, and various anti-IL23 and anti-TNFα antibodies of the present disclosure, and their sequence identifiers. The sequences also are included in the accompanying Sequence Listing.

TABLE 1
Description of exemplary antibody sequences

| SEQ | | |
|---|---|---|
| ID | | |
| NO: | SEQUENCE | DESCRIPTION |
| 1 | MHPQQLVISWFSLVLLASPLMAIWELEKDVYVVELDWHPD | Recombinant Canine IL23 |
| | APGEMVVLTCHTPEEDDITWTSAQSSEVLGSGKTLTIQVK | interleukin-12 subunit beta |
| | EFGDAGQYTCHKGGKVLSRSLLLIHKKEDGIWSTDILKEQ | isoform X1 [Canis lupus |
| | KESKNKIFLKCEAKNYSGRFTCWWLTAISTDLKFSVKSSR | familiaris] |
| | GFSDPQGVTCGAVTLSAERVRVDNRDYKKYTVECQEGSAC | p40 +p19 |
| | PSAEESLPIEVVVDAIHKLKYENYTSSFFIRDIIKPDPPT | >XP_003988941.1 |
| | NLQLKPLKNSRHVEVSWEYPDTWSTPHSYFSLTFCIQAQG | interleukin-23 subunit alpha |
| | KNNREKKDRLCVDKTSAKVVCHKDAKIRVQARDRYYSSSW | isoform X3 [Felis catus] |
| | SDWASVSCSGSGSSRGGSGSGGSGGGGSKLRAVPEGSSPA | Note: Avitag (in red) for |
| | WIQGQQLSQKLCTLVWSAHPPMGHVDLPREEGDGETTSDV | Biotinylation for Biosensor |
| | PRIQCGDGCDPQGLKDNSQFCLQRIHQGLVFYEKLLGSDI | analysis |
| | FTGEPSLLPDGPVGQLHANLLGLRQLLQPEGHHWETEQTP | |
| | SPSPSQPWQRLLLRLKILRSLQAFVAVAARVFAHGAATLS | |
| | PGSGSGLNDIFEAQKIEWHEHHHHHHHHHH | |
| 2 | MHPQQLVIAWFSLVLLAPPLMAIWELEKNVYVVELDWHPD | Recombinant feline IL23 |
| | APGEMVVLTCDTPEEDDITWTSDQSSEVLGSGKTLTIQVK | >AAB93835.1 interleukin- |
| | EFADAGQYTCHKGGEVLSHSFLLIHKKEDGIWSTDILREQ | 12 p40 subunit [Felis catus] |
| | KESKNKIFLKCEAKNYSGRFTCWWLTAISTDLKFTVKSSR | p40 +p19 |
| | GSSDPQGVTCGAATLSAEKVRVDNRDYKKYTVECQEGSAC | Note: Avitag (in red) for |
| | PAAEESLPIEVVVDAIHKLKYENYTSSFFIRDIIKPDPPK | Biotinylation for Biosensor |
| | NLQLKPLKNSRHVEVSWEYPDTWSTPHSYFSLTFGVQVQG | analysis |
| | KNNREKKDRLSVDKTSAKVVCHKDAKIRVQARDRYYSSSW | |
| | SNWASVSCSGSGSSRGGSGSGGSGGGGSKLRAVPEGSSPA | |
| | WALGQQLSQKLCTLAWSAHPPTGHVDLPREQGDDETTDDV | |
| | PHIQCGDGCDPQGLRDNSQFCLQRIHQGLVFYEKLLGSDI | |
| | FTGEPSLLPDSPVGQLHASLLGLRQLLQPDGQHWETEQTP | |
| | NPSPNQPWQRLLLRFKILRSLQAFVAVAARVFAHGAATLS | |
| | PGSGSGLNDIFEAQKIEWHEHHHHHHHHHH | |
| 3 | DIQMTQSPSSLSASVGDRVTITCRASQGISSWLAWYQQKP | Anti-IL23 C340 |
| | EKAPKSLIYAASSLQSGVPSRFSGSGSGTDFTLTISSLQP | VL domain |
| | EDFATYYCQQYNIYPYTFGQGTKLEIK | |
| 4 | RASQGISSWLA | Anti-IL23 clone C340 |
| | | HVR-L1 |
| 5 | YAASSLQS | Anti-IL23 clone C340 |
| | | HVR-L2 |
| 6 | QQYNIYPYT | Anti-IL23 clone C340 |
| | | HVR-L3 |
| 7 | EVQLVQSGAEVKKPGESLKISCKGSGYSFTTYWLGWVRQM | Anti-IL23 C340 |
| | PGKGLDWIGIMSPVDSDIRYSPSFQGQVTMSVDKSITTAY | VH domain |
| | LQWNSLKASDTAMYYCARRRPGQGYFDFWGQGTLVTVSS | |
| 8 | KGSGYSFTTYWLG | Anti-IL23 clone C340 |
| | | HVR-H1 |
| 9 | IMSPVDSDIR | Anti-IL23 clone C340 |
| | | HVR-H2 |
| 10 | ARRRPGQGYFDF | Anti-IL23 clone C340 |
| | | HVR-H3 |
| 11 | DIVMTQTPLSLSVSPGEPASISCRASQGISSWLAWYQQKA | Anti-IL23 |
| | EKAPKSLIYAASSLQSGVPDRFSGSGSGTDFTLRIGRVEA | Caninized VL domain V2.1 |
| | EDAGIYYCQQYNIYPYTFGQGTRLEVR | |
| 12 | EVQLVQSGAEVKKPGASVKVSCKGSGYSFTTYWLGWVRQA | Anti-IL23 |
| | PGAGLDWMGIMSPVDSDIRYSPSFQGQVTMSVDTSTSTAY | Caninized VH domain V1.1 |
| | MELSSLRAGDIAVYYCARRRPGQGYFDFWGQGTLVTVSS | |
| 13 | DIVMTQSPASLSLSQEEKVTITCRASQGISSWLAWYQQKP | Anti-IL23 C340 |
| | EKAPKSLIYAASSLQSGVPSRFSGSGSGTDFSFTISSLEP | Caninized VL domain (VL- |
| | EDVAVYYCQQYNIYPYTFGQGTKLEIK | A) |
| 14 | EVQLVESGGDLVKPGGSLRLSCKGSGYSFTTYWLGWVRQM | Anti-IL23 C340 |
| | PGKGLDWIGIMSPVDSDIRYSPSFQGRFTISRDNAKNTVY | Caninized VH domain |
| | LQMNSLRAEDTAMYYCARRRPGQGYFDFWGQGTLVTVSS | (VH-A) |
| 15 | EIQMTQSPSSLSASPGDRVTITCRASQGISSWLAWYQQKP | Anti-IL23 C340 |
| | EKAPKSLIYAASSLQSGVPSRFSGSGSGTDFTLTISSLEP | Felinized VL domain |
| | EDAATYYCQQYNIYPYTFGQGTKLEIK | |
| 16 | QVLLVQSGAEVRKPEASVKIFCKGSGYSFTTYWLGWVRQA | Anti-IL23 C340 |
| | PGQGLDWMGIMSPVDSDIRYAQNFQGKLTLTVDTSTNTAY | Felinized VH domain |
| | MELRNLRSTDTAVYYCARRRPGQGYFDFWGQGTLVTVSS | |
| 17 | DIQMTQSPSSLSASVGDRVTITCRASQGISSWLAWYQQKP | Anti-IL23 Chimeric |
| | EKAPKSLIYAASSLQSGVPSRFSGSGSGTDFTLTISSLQP | C340 VL domain |
| | EDFATYYCQQYNIYPYTFGQGTKLEIKRNDAQPAVYLFQP | +canine kappa light chain |
| | SPDQLHTGSASVVCLLNSFYPKDINVKWKVDGVIQDTGIQ | constant region |
| | ESVTEQDKDSTYSLSSTLTMSSTEYLSHELYSCEITHKSL | |
| | PSTLIKSFQRSECQRVD | |
| 18 | EVQLVQSGAEVKKPGESLKISCKGSGYSFTTYWLGWVRQM | Anti-IL23 Chimeric |
| | PGKGLDWIGIMSPVDSDIRYSPSFQGQVTMSVDKSITTAY | C340 VH domain |
| | LQWNSLKASDTAMYYCARRRPGQGYFDFWGQGTLVTVSSA | +canine IgG-B constant |
| | STTAPSVFPLAPSCGSTSGSTVALACLVSGYFPEPVTVSW | region |
| | NSGSLTSGVHTFPSVLQSSGLYSLSSMVTVPSSRWPSETF | |
| | TCNVAHPASKTKVDKPVPKRENGRVPRPPDCPKCPAPEML | |
| | GGPSVFIFPPKPKDTLYIARTPEVTCVVVDLDPEDPEVQI | |
| | SWFVDGKQMQTAKTQPREEQFNGTYRVVSVLPIGHQDWLK | |
| | GKQFTCKVNNKALPSPIERTISKARGQAHQPSVYVLPPSR | |
| | EELSKNTVSLTCLIKDFFPPDIDVEWQSNGQQEPESKYRT | |
| | TPPQLDEDGSYFLYSKLSVDKSRWQRGDTFICAVMHEALH | |
| | NHYTQESLSHSPGK | |
| 19 | DIVMTQTPLSLSVSPGEPASISCRASQGISSWLAWYQQKA | Anti-IL23 C340 |
| | EKAPKSLIYAASSLQSGVPDRFSGSGSGTDFTLRIGRVEA | Caninized VL domain V2.1 |
| | EDAGIYYCQQYNIYPYTFGQGTRLEVRRNDAQPAVYLFQP | +canine kappa light chain |
| | SPDQLHTGSASVVCLLNSFYPKDINVKWKVDGVIQDTGIQ | constant region |
| | ESVTEQDKDSTYSLSSTLTMSSTEYLSHELYSCEITHKSL | |
| | PSTLIKSFQRSECQRVD | |
| 20 | EVQLVQSGAEVKKPGASVKVSCKGSGYSFTTYWLGWVRQA | Anti-IL23 |
| | PGAGLDWMGIMSPVDSDIRYSPSFQGQVTMSVDTSTSTAY | Caninized VH domain V1.1 |
| | MELSSLRAGDIAVYYCARRRPGQGYFDFWGQGTLVTVSSA | + Fc IgG-B constant |
| | STTAPSVFPLAPSCGSTSGSTVALACLVSGYFPEPVTVSW | region (long-acting L252Y |
| | NSGSLTSGVHTFPSVLQSSGLYSLSSMVTVPSSRWPSETF | variant) |
| | TCNVAHPASKTKVDKPVPKRENGRVPRPPDCPKCPAPEML | |
| | GGPSVFIFPPKPKDTLYIARTPEVTCVVVDLDPEDPEVQI | |
| | SWFVDGKQMQTAKTQPREEQFNGTYRVVSVLPIGHQDWLK | |
| | GKQFTCKVNNKALPSPIERTISKARGQAHQPSVYVLPPSR | |
| | EELSKNTVSLTCLIKDFFPPDIDVEWQSNGQQEPESKYRT | |
| | TPPQLDEDGSYFLYSKLSVDKSRWQRGDTFICAVMHEALH | |
| | NHYTQESLSHSPGK | |
| 21 | EVQLVQSGAEVKKPGASVKVSCKGSGYSFTTYWLGWVRQA | Anti-IL23 |
| | PGAGLDWMGIMSPVDSDIRYSPSFQGQVTMSVDTSTSTAY | Caninized VH domain V1.1 |
| | MELSSLRAGDIAVYYCARRRPGQGYFDFWGQGTLVTVSSA | + Fc IgG-B (long-acting |
| | STTAPSVFPLAPSCGSTSGSTVALACLVSGYFPEPVTVSW | L252Y variant) + Knob |
| | NSGSLTSGVHTFPSVLQSSGLYSLSSMVTVPSSRWPSETF | |
| | TCNVAHPASKTKVDKPVPKRENGRVPRPPDCPKCPAPEML | |
| | GGPSVFIFPPKPKDTLYIARTPEVTCVVVDLDPEDPEVQI | |
| | SWFVDGKQMQTAKTQPREEQFNGTYRVVSVLPIGHQDWLK | |
| | GKQFTCKVNNKALPSPIERTISKARGQAHQPSVYVLPPSR | |
| | EELSKNTVSLÆCLIKDFFPPDIDVEWQSNGQQEPESKYRT | |
| | TPPQLDEDGSYFLYSKLSVDKSRWQRGDTFICAVMHEALH | |
| | NHYTQESLSHSPGK | |
| 22 | DIVMTQSPASLSLSQEEKVTITCRASQGISSWLAWYQQKP | Anti-IL23 C340 |
| | EKAPKSLIYAASSLQSGVPSRFSGSGSGTDFSFTISSLEP | Caninized VL domain (VL- |
| | EDVAVYYCQQYNIYPYTFGQGTKLEIKRNDAQPAVYLFQP | A) |
| | SPDQLHTGSASVVCLLNSFYPKDINVKWKVDGVIQDTGIQ | +canine kappa light chain |
| | ESVTEQDKDSTYSLSSTLTMSSTEYLSHELYSCEITHKSL | constant region |
| | PSTLIKSFQRSECQRVD | |
| 23 | EVQLVESGGDLVKPGGSLRLSCKGSGYSFTTYWLGWVRQM | Anti-IL23 |
| | PGKGLDWIGIMSPVDSDIRYSPSFQGRFTISRDNAKNTVY | Caninized VH domain |
| | LQMNSLRAEDTAMYYCARRRPGQGYFDFWGQGTLVTVSSA | (VH-A) |
| | STTAPSVFPLAPSCGSTSGSTVALACLVSGYFPEPVTVSW | + Fc IgG-B (long-acting |
| | NSGSLTSGVHTFPSVLQSSGLYSLSSMVTVPSSRWPSETF | L252Y variant) |
| | TCNVAHPASKTKVDKPVPKRENGRVPRPPDCPKCPAPEML | |
| | GGPSVFIFPPKPKDTLYIARTPEVTCVVVDLDPEDPEVQI | |
| | SWFVDGKQMQTAKTQPREEQFNGTYRVVSVLPIGHQDWLK | |
| | GKQFTCKVNNKALPSPIERTISKARGQAHQPSVYVLPPSR | |
| | EELSKNTVSLTCLIKDFFPPDIDVEWQSNGQQEPESKYRT | |
| | TPPQLDEDGSYFLYSKLSVDKSRWQRGDTFICAVMHEALH | |
| | NHYTQESLSHSPGK | |
| 24 | EIQMTQSPSSLSASPGDRVTITCRASQGISSWLAWYQQKP | Anti-IL23 C340 |
| | EKAPKSLIYAASSLQSGVPSRFSGSGSGTDFTLTISSLEP | Felinized VL domain |
| | EDAATYYCQQYNIYPYTFGQGTKLEIKRSDAQPSVFLFQP | +feline kappa light chain |
| | SLDELHTGSASIVCILNDFYPKEVNVKWKVDGVVQNKGIQ | constant region |
| | ESTTEQNSKDSTYSLSSTLTMSSTEYQSHEKFSCEVTHKS | |
| | LASTLVKSFNRSECQRE | |
| 25 | QVLLVQSGAEVRKPEASVKIFCKGSGYSFTTYWLGWVRQA | Anti-IL23 |
| | PGQGLDWMGIMSPVDSDIRYAQNFQGKLTLTVDTSTNTAY | Felinized VH domain |
| | MELRNLRSTDTAVYYCARRRPGQGYFDFWGQGTLVTVSSA | + feline Fc IgG1 (long- |
| | STTAPSVFPLAPSCGTTSGATVALACLVLGYFPEPVTVSW | acting S252Y variant) |
| | NSGALTSGVHTFPAVLQASGLYSLSSMVTVPSSRWLSDTF | |
| | TCNVAHPPSNTKVDKTVRKTDHPPGPKPCDCPKCPPPEML | |
| | GGPSIFIFPPKPKDTLYISRTPEVTCLVVDLGPDDSDVQI | |
| | TWFVDNTQVYTAKTSPREEQFNSTYRVVSVLPILHQDWLK | |
| | GKEFKCKVNSKSLPSPIERTISKAKGQPHEPQVYVLPPAQ | |
| | EELSRNKVSVTCLIKSFHPPDIAVEWEITGQPEPENNYRT | |
| | TPPQLDSDGTYFVYSKLSVDRSHWQRGNTYTCSVSHEALH | |
| | SHHTQKSLTQSPGK | |
| 26 | DIQMTQSPSSLSASVGDRVTITCRASQGIRNYLAWYQQKP | Anti-TNFα D2E7 |
| | GKAPKLLIYAASTLQSGVPSRFSGSGSGTDFTLTISSLQP | VL domain |
| | EDVATYYCQRYNRAPYTFGQGTKVEIK | |
| 27 | RASQGIRNYLA | Anti-TNFα D2E7 |
| | | HVR-L1 |
| 28 | AASTLQ | Anti-TNFα D2E7 |
| | | HVR-L2 |
| 29 | QRYNRAPYT | Anti-TNFα D2E7 |
| | | HVR-L3 |
| 30 | EVQLVESGGGLVQPGRSLRLSCAASGFTFDDYAMHWVRQA | Anti-TNFα D2E7 |
| | PGKGLEWVSAITWNSGHIDYADSVEGRFTISRDNAKNSLY | VH domain |
| | LQMNSLRAEDTAVYYCAKVSYLSTASSLDYWGQGTLVTVS | |
| | S | |
| 31 | FTFDDYAMH | Anti-TNFα D2E7 |
| | | HVR-H1 |
| 32 | AITWNSGHIDYADSVEGR | Anti-TNFα D2E7 |
| | | HVR-H2 |
| 33 | AKVSYLSTASSLDY | Anti-TNFα D2E7 |
| | | HVR-H3 |
| 34 | DIVMTQTPLSLSVSPGEPASISCRASQGIRNYLAWYQQKP | Anti-TNFα D2E7 |
| | GKAPKLLIYAASTLQSGVPDRFSGSGSGTD | Caninized VL domain |
| | FTLRIGRVEAEDAGIYYCQRYNRAPYTFGQGTRLEVR | |
| 35 | EVQLVESGGDLVKPAESLRLSCVASGFTFDDYAMHWVRQA | Anti-TNFα D2E7 |
| | PGKGLEWVSAITWNSGHIDYADSVEGRFTISRDNARNTLY | Caninized VH domain |
| | LQMNSLRAEDTAVYYCAKVSYLSTASSLDYWGQGTLVTVS | |
| | S | |
| 36 | DVVMTQTPLSLAVTPGEPASISCRASQGIRNYLAWYQQKP | Anti-TNFα D2E7 |
| | GKAPKLLIYAASTLQSGVPDRFSGSGSGTDFTLRISRVEA | Felinized VL domain |
| | DDVGVYYCQRYNRAPYTFGQGTKLEIR | |
| 37 | DVQLVESGGDLVKPGGSQRLTCVASGFTFDDYAMHWVRQA | Anti-TNFα D2E7 |
| | PGKGLEWVSAITWNSGHIDYADSVEGRFTISRDNAKNTLY | Felinized VH domain |
| | LQMNSLRTEDTATYYCAKVSYLSTASSLDYWGQGALVTVS | |
| | S | |
| 38 | DIQMTQSPSSLSASVGDRVTITCRASQGIRNYLAWYQQKP | Anti-TNFα Chimeric D2E7 |
| | GKAPKLLIYAASTLQSGVPSRFSGSGSGTDFTLTISSLQP | VL domain |
| | EDVATYYCQRYNRAPYTFGQGTKVEIKRNDAQPAVYLFQP | + canine kappa light |
| | SPDQLHTGSASVVCLLNSFYPKDINVKWKVDGVIQDTGIQ | constant region |
| | ESVTEQDKDSTYSLSSTLTMSSTEYLSHELYSCEITHKSL | |
| | PSTLIKSFQRSECQRVD | |
| 39 | EVQLVESGGGLVQPGRSLRLSCAASGFTFDDYAMHWVRQA | Anti-TNFα Chimeric D2E7 |
| | PGKGLEWVSAITWNSGHIDYADSVEGRETISRDNAKNSLY | VH domain |
| | LQMNSLRAEDTAVYYCAKVSYLSTASSLDYWGQGTLVTVS | + canine IgG-B constant |
| | SASTTAPSVFPLAPSCGSTSGSTVALACLVSGYFPEPVTV | region |
| | SWNSGSLTSGVHTFPSVLQSSGLYSLSSMVTVPSSRWPSE | |
| | TFTCNVAHPASKTKVDKPVPKRENGRVPRPPDCPKCPAPE | |
| | MLGGPSVFIFPPKPKDTLLIARTPEVTCVVVDLDPEDPEV | |
| | QISWFVDGKQMQTAKTQPREEQFNGTYRVVSVLPIGHQDW | |
| | LKGKQFTCKVNNKALPSPIERTISKARGQAHQPSVYVLPP | |
| | SREELSKNTVSLTCLIKDFFPPDIDVEWQSNGQQEPESKY | |
| | RTTPPQLDEDGSYFLYSKLSVDKSRWQRGDTFICAVMHEA | |
| | LHNHYTQESLSHSPGK | |
| 40 | DIVMTQTPLSLSVSPGEPASISCRASQGIRNYLAWYQQKP | Anti-TNFα D2E7 |
| | GKAPKLLIYAASTLQSGVPDRFSGSGSGTDFTLRIGRVEA | Caninized VL domain |
| | EDAGIYYCQRYNRAPYTFGQGTRLEVRRNDAQPAVYLFQP | + canine kappa light |
| | SPDQLHTGSASVVCLLNSFYPKDINVKWKVDGVIQDTGIQ | constant region |
| | ESVTEQDKDSTYSLSSTLTMSSTEYLSHELYSCEITHKSL | |
| | PSTLIKSFQRSECQRVD | |
| 41 | DIVMTQTPLSLSVSPGEPASISCRASQGIRNYLAWYQQKP | Anti-TNFα D2E7 |
| | GCAPKLLIYAASTLQSGVPDRFSGSGSGTDFTLRIGRVEA | Caninized VL domain |
| | EDAGIYYCQRYNRAPYTFGQGTRLEVRRNDAQPAVYLFQP | +canine kappa light chain |
| | SPDQLHTGSASVVCLLNSFYPKDINVKWKVDGVIQDTGIQ | constant region + Lock |
| | ESVTEQDKDSTYSLSSTLTMSSTEYLSHELYSCEITHKSL | |
| | PSTLIKSFQRSESQRVD | |
| 42 | EVQLVESGGDLVKPAESLRLSCVASGFTFDDYAMHWVRQA | Anti-TNFα D2E7 |
| | PGKGLEWVSAITWNSGHIDYADSVEGRETISRDNARNTLY | Caninized VH domain |
| | LQMNSLRAEDTAVYYCAKVSYLSTASSLDYWGQGTLVTVS | + canine IgG-B constant |
| | SASTTAPSVFPLAPSCGSTSGSTVALACLVSGYFPEPVTV | region |
| | SWNSGSLTSGVHTFPSVLQSSGLYSLSSMVTVPSSRWPSE | |
| | TFTCNVAHPASKTKVDKPVPKRENGRVPRPPDCPKCPAPE | |
| | MLGGPSVFIFPPKPKDTLLIARTPEVTCVVVDLDPEDPEV | |
| | QISWFVDGKQMQTAKTQPREEQFNGTYRVVSVLPIGHQDW | |
| | LKGKQFTCKVNNKALPSPIERTISKARGQAHQPSVYVLPP | |
| | SREELSKNTVSLTCLIKDFFPPDIDVEWQSNGQQEPESKY | |
| | RTTPPQLDEDGSYFLYSKLSVDKSRWQRGDTFICAVMHEA | |
| | LHNHYTQESLSHSPGK | |
| 43 | EVQLVESGGDLVKPAESLRLSCVASGFTFDDYAMHWVRQA | Anti-TNFα D2E7 |
| | PGKGLEWVSAITWNSGHIDYADSVEGRFTISRDNARNTLY | Caninized VH domain |
| | LQMNSLRAEDTAVYYCAKVSYLSTASSLDYWGQGTLVTVS | + canine IgG-B constant |
| | SASTTAPSVFPLAPSCGSTSGSTVALACLVSGYFPEPVTV | region (long-acting L252Y |
| | SWNSGSLTSGVHTFPSVLQSSGLYSLSSMVTVPSSRWPSE | variant) |
| | TFTCNVAHPASKTKVDKPVPKRENGRVPRPPDCPKCPAPE | |
| | MLGGPSVFIFPPKPKDTLYIARTPEVTCVVVDLDPEDPEV | |
| | QISWFVDGKQMQTAKTQPREEQFNGTYRVVSVLPIGHQDW | |
| | LKGKQFTCKVNNKALPSPIERTISKARGQAHQPSVYVLPP | |
| | SREELSKNTVSLTCLIKDFFPPDIDVEWQSNGQQEPESKY | |
| | RTTPPQLDEDGSYFLYSKLSVDKSRWQRGDTFICAVMHEA | |
| | LHNHYTQESLSHSPGK | |
| 44 | EVQLVESGGDLVKPAESLRLSCVASGFTFDDYAMHWVRQA | Anti-TNFα D2E7 |
| | PGKCLEWVSAITWNSGHIDYADSVEGRETISRDNARNTLY | Caninized VH domain |
| | LQMNSLRAEDTAVYYCAKVSYLSTASSLDYWGQGTLVTVS | + canine IgG-B constant |
| | SASTTAPSVFPLAPSSGSTSGSTVALACLVSGYFPEPVTV | region (long-acting L252Y |
| | SWNSGSLTSGVHTFPSVLQSSGLYSLSSMVTVPSSRWPSE | variant) + Hole |
| | TFTCNVAHPASKTKVDKPVPKRENGRVPRPPDCPKCPAPE | |
| | MLGGPSVFIFPPKPKDTLXIARTPEVTCVVVDLDPEDPEV | |
| | QISWFVDGKQMQTAKTQPREEQFNGTYRVVSVLPIGHQDW | |
| | LKGKQFTCKVNNKALPSPIERTISKARGQAHQPSVYVLPP | |
| | SREELSKNTVSLSCAIKDFFPPDIDVEWQSNGQQEPESKY | |
| | RTTPPQLDEDGSYFLASKLSVDKSRWQRGDTFICAVMHEA | |
| | LHNHYTQESLSHSPG | |
| 45 | EVQLVESGGDLVKPAESLRLSCVASGFTFDDYAMHWVRQA | Anti-TNFα scFv (ver.1) |
| | PGKGLEWVSAITWNSGHIDYADSVEGRFTISRDNARNTLY | Caninized VH and |
| | LQMNSLRAEDTAVYYCAKVSYLSTASSLDYWGQGTLVTVS | Caninized VL domains |
| | SGGGGGSGGGGSGGGGGSDIVMTQTPLSLSVSPGEPASIS | |
| | CRASQGIRNYLAWYQQKPGKAPKLLIYAASTLQSGVPDRF | |
| | SGSGSGTDFTLRIGRVEAEDAGIYYCQRYNRAPYTFGQGT | |
| | RLEVR | |
| 46 | EVQLVESGGDLVKPAESLRLSCVASGFTFDDYAMHWVRQA | Anti-TNFα scFv (ver.2) |
| | PGKCLEWVSAITWNSGHIDYADSVEGRFTISRDNARNTLY | Caninized VH and |
| | LQMNSLRAEDTAVYYCAKVSYLSTASSLDYWGQGTLVTVS | Caninized VL domains |
| | SGGGGGSGGGGSGGGGGSDIVMTQTPLSLSVSPGEPASIS | + double C variants for S-S |
| | CRASQGIRNYLAWYQQKPGKAPKLLIYAASTLQSGVPDRE | bond formation |
| | SGSGSGTDFTLRIGRVEAEDAGIYYCQRYNRAPYTFGCGT | |
| | RLEVR | |
| 47 | EVQLVQSGAEVKKPGASVKVSCKGSGYSFTTYWLGWVRQA | Bispecific anti-IL23/anti- |
| | PGAGLDWMGIMSPVDSDIRYSPSFQGQVTMSVDTSTSTAY | TNFα antibody |
| | MELSSLRAGDIAVYYCARRRPGQGYFDFWGQGTLVTVSSA | Anti-IL23 Caninized VH |
| | STTAPSVFPLAPSCGSTSGSTVALACLVSGYFPEPVTVSW | domain V1.1 |
| | NSGSLTSGVHTFPSVLQSSGLYSLSSMVTVPSSRWPSETF | + Fc IgGB (long-acting |
| | TCNVAHPASKTKVDKPVPKRENGRVPRPPDCPKCPAPEML | L252Y variant) |
| | GGPSVFIFPPKPKDTLYIARTPEVTCVVVDLDPEDPEVQI | + Anti-TNFα scFv (ver.1) |
| | SWFVDGKQMQTAKTQPREEQFNGTYRVVSVLPIGHQDWLK | with Caninized VH and |
| | GKQFTCKVNNKALPSPIERTISKARGQAHQPSVYVLPPSR | caninized VL domains |
| | EELSKNTVSLTCLIKDFFPPDIDVEWQSNGQQEPESKYRT | |
| | TPPQLDEDGSYFLYSKLSVDKSRWQRGDTFICAVMHEALH | |
| | NHYTQESLSHSPGGGGGSGGGEVQLVESGGDLVKPAESLR | |
| | LSCVASGFTFDDYAMHWVRQAPGKGLEWVSAITWNSGHID | |
| | YADSVEGRFTISRDNARNTLYLQMNSLRAEDTAVYYCAKV | |
| | SYLSTASSLDYWGQGTLVTVSSGGGGGSGGGGSGGGGGSD | |
| | IVMTQTPLSLSVSPGEPASISCRASQGIRNYLAWYQQKPG | |
| | KAPKLLIYAASTLQSGVPDRFSGSGSGTDFTLRIGRVEAE | |
| | DAGIYYCQRYNRAPYTFGQGTRLEVR | |
| 48 | EVQLVQSGAEVKKPGASVKVSCKGSGYSFTTYWLGWVRQA | Bispecific anti-IL23/anti- |
| | PGAGLDWMGIMSPVDSDIRYSPSFQGQVTMSVDTSTSTAY | TNFα antibody |
| | MELSSLRAGDIAVYYCARRRPGQGYFDFWGQGTLVTVSSA | Anti-IL23 with Caninized |
| | STTAPSVFPLAPSCGSTSGSTVALACLVSGYFPEPVTVSW | VH domain V1.1 |
| | NSGSLTSGVHTFPSVLQSSGLYSLSSMVTVPSSRWPSETF | + Fc IgGB (long-acting |
| | TCNVAHPASKTKVDKPVPKRENGRVPRPPDCPKCPAPEML | L252Y variant) |
| | GGPSVFIFPPKPKDTLYIARTPEVTCVVVDLDPEDPEVQI | + Anti-TNFα scFv (ver.2) |
| | SWFVDGKQMQTAKTQPREEQFNGTYRVVSVLPIGHQDWLK | with Caninized VH and |
| | GKQFTCKVNNKALPSPIERTISKARGQAHQPSVYVLPPSR | Caninized VL domains |
| | EELSKNTVSLTCLIKDFFPPDIDVEWQSNGQQEPESKYRT | (with double C variants for |
| | TPPQLDEDGSYFLYSKLSVDKSRWQRGDTFICAVMHEALH | S-S bond formation) |
| | NHYTQESLSHSPGGGGGSGGGEVQLVESGGDLVKPAESLR | |
| | LSCVASGFTFDDYAMHWVRQAPGKCLEWVSAITWNSGHID | |
| | YADSVEGRETISRDNARNTLYLQMNSLRAEDTAVYYCAKV | |
| | SYLSTASSLDYWGQGTLVTVSSGGGGGSGGGGSGGGGGSD | |
| | IVMTQTPLSLSVSPGEPASISCRASQGIRNYLAWYQQKPG | |
| | KAPKLLIYAASTLQSGVPDRFSGSGSGTDFTLRIGRVEAE | |
| | DAGIYYCQRYNRAPYTFGCGTRLEVR | |

Novel antibodies directed against canine, feline, or equine IL23 and/or canine, feline, or equine TNFα are provided herein. The anti-IL23 and/or anti-TNFα antibodies provided herein include, but are not limited to, monoclonal antibodies, chimeric antibodies, caninized, felinized, or equinized antibodies, scFv antibodies, and bispecific antibodies that bind to both IL23 and TNFα. Also provided herein are amino acid sequences of monoclonal antibodies. For example, the light and heavy chain hypervariable regions (HVR-L1, HVR-L2, HVR-L3, HVR-H1, HVR-H2, HVR-H3), the heavy chain variable domain (VH), the light chain variable domain (VL), the variable region heavy chain framework sequences, and the variable region light chain framework sequences for monoclonal antibodies described herein are provided. In addition, the amino acid sequences of HVRs, framework sequences, light chain variable domain (VL) sequences, and heavy chain variable domain (VH) sequences of different caninized, and felinized light and heavy chains are provided.

The present disclosure provides a novel anti-IL23 antibody of the present disclosure is the monoclonal antibody referred to as “clone 340” or “C340.” As shown in Table 1, the amino acid sequences of the C340 light chain variable domain VL is provided as SEQ ID NO: 3, and the C340 heavy chain variable domain VH is provided as SEQ ID NO: 7. The corresponding hypervariable regions of the C340 VL domain, HVR-L1, HVR-L2, HVR-L3, are provided as SEQ ID NO: 4, 5, and 6, respectively. The corresponding hypervariable regions of the C340 VH domain, HVR-H1, HVR-H2, HVR-H3, are provided as SEQ ID NO: 8, 9, and 10, respectively.

In at least one embodiment, the present disclosure provides chimeric anti-IL23 antibodies derived from C340. In one embodiment of a chimeric anti-IL23 antibody, the antibody comprises the VL and VH domains of SEQ ID NO: 3 and 7, respectively. The VL domain of the chimera is linked with the canine kappa light chain constant region and the VH domain of the chimera is linked with canine IgG-B constant region. The light chain and heavy chain amino acid sequences of these anti-IL23 chimeric antibodies are provided as SEQ ID NO: 17 and 18, respectively. The tight binding affinity of the anti-IL23 chimeric antibodies to canine IL23 was measured as described in the Examples.

In at least one embodiment, the present disclosure provides caninized versions of the C340 anti-IL23 antibody. Two different caninized versions of the VL domain are provided as SEQ ID NO: 11 and 13. Two different caninized versions of the VH domain are provided as SEQ ID NO: 12 and 14. The caninized versions of the VL domain comprise the C340 VL domain, HVR-L1, HVR-L2, HVR-L3, sequences of SEQ ID NO: 4, 5, and 6, respectively. The caninized versions of VH domain comprise the C340 VH domain, HVR-H1, HVR-H2, HVR-H3 sequences of SEQ ID NO: 8, 9, and 10, respectively.

Exemplary caninized anti-IL23 antibody light chain (LC) and heavy chain (HC) sequences comprising the caninized VL domains of SEQ ID NO: 11 and 13, and the caninized VH domains of SEQ ID NO: 12 and 14, are provided in Table 1. Exemplary caninized anti-IL23 light chains comprising the canine kappa light chain constant region are SEQ ID NO: 19, and 22. Exemplary caninized anti-IL23 heavy chains comprising the canine Fc IgG-B constant region are provided as SEQ ID NO: 20, 21 and 23. It is contemplated that a range of exemplary caninized anti-IL23 antibodies can be prepared using any combination of the LC sequences of SEQ ID NO: 19 and 22, with the HC sequences of SEQ ID NO: 20, 21, and 23.

In at least one embodiment, the present disclosure also provides felinized versions of the C340 anti-IL23 antibody. An exemplary felinized version of the VL domain is provided as SEQ ID NO: 15, a felinized versions of the VH domain is provided as SEQ ID NO: 16. The felinized version of the VL domain of SEQ ID NO: 15 comprises the C340 HVR-L1, HVR-L2, HVR-L3 sequences of SEQ ID NO: 4, 5, and 6, respectively. Similarly, the felinized version of the VH domain of SEQ ID NO: 16, comprises the C340 HVR-H1, HVR-H2, HVR-H3 sequences of SEQ ID NO: 8, 9, and 10, respectively.

The present disclosure provides a novel anti-TNFα antibody referred to as “D2E7” that specifically binds canine, feline, and/or equine TNFα. As shown in Table 1, the amino acid sequences of the D2E7 anti-TNFα antibody light chain variable domain VL is provided as SEQ ID NO: 26, and the C340 heavy chain variable domain VH is provided as SEQ ID NO: 30. The corresponding hypervariable regions of the D2E7 VL domain, HVR-L1, HVR-L2, HVR-L3, are provided as SEQ ID NO: 27, 28, and 29, respectively. The corresponding hypervariable regions of the D2E7 VH domain, HVR-H1, HVR-H2, HVR-H3, are provided as SEQ ID NO: 31, 32, and 33, respectively.

In at least one embodiment, the present disclosure provides chimeric anti-TNFα antibodies derived from the exemplary anti-TNFα monoclonal antibody, D2E7. In at least one embodiment, the chimeric anti-TNFα D2E7 antibodies comprise the VL domain and VH domain of SEQ ID NO: 26 and 30, respectively, linked to the canine kappa light constant region and the canine IgG-B constant region, respectively. Exemplary light chain (LC) and heavy chain (HC) amino acid sequences of a chimeric anti-TNFα D2E7 antibodies are provided in Table 1 as SEQ ID NO: 38 and 39, respectively.

In at least one embodiment, the present disclosure provides caninized versions of the anti-TNFα D2E7 antibody VL domain and VH domain as SEQ ID NO: 34 and 35, respectively. Exemplary caninized anti-TNFα antibody light chain (LC) and heavy chain (HC) sequences comprising the caninized VL domain of SEQ ID NO: 34, and the caninized VH domain of SEQ ID NO: 35, are provided in Table 1. Exemplary caninized anti-TNFα light chains comprising the canine kappa light chain constant region are provided as SEQ ID NO: 40 and 41. Exemplary caninized anti-TNFα heavy chains comprising the canine Fc IgG-B constant region are provided as SEQ ID NO: 42, 43, and 44. It is contemplated that a range of exemplary caninized anti-TNFα antibodies can be prepared using any combination of the LC sequences of SEQ ID NO: 40 and 41, with the HC sequences of SEQ ID NO: 42, 43, and 44.

In at least one embodiment, the present disclosure also provides felinized versions of the D2E7 anti-TNFα antibody. An exemplary felinized version of the VL domain is provided as SEQ ID NO: 36, and an exemplary felinized versions of the VH domain is provided as SEQ ID NO: 37. The felinized version of the VL domain of SEQ ID NO: 36 comprises the D2E7 HVR-L1, HVR-L2, HVR-L3 sequences of SEQ ID NO: 27, 28, and 29, respectively. Similarly, the felinized version of the VH domain of SEQ ID NO: 37, comprises the D2E7 HVR-H1, HVR-H2, HVR-H3 sequences of SEQ ID NO: 31, 32, and 33, respectively.

In at least one embodiment, the present disclosure provides an anti-TNFα scFv antibody with a VL domain comprising the D2E7 HVR-L1, HVR-L2, HVR-L3 sequences of SEQ ID NO: 27, 28, and 29, respectively, fused via a polypeptide linker to a VH domain comprising the D2E7 HVR-H1, HVR-H2, HVR-H3 sequences of SEQ ID NO: 31, 32, and 33, respectively. In at least one embodiment, the scFv antibody VL domain comprises an amino acid sequence of SEQ ID NO: 26, 34, or 40, and the scFv antibody VL domain comprises an amino acid sequence of SEQ ID NO: 30, 35, or 41. In at least one embodiment, the polypeptide linker that fuses the scFv antibody VL and VH domains comprises the amino acid sequence GGGGGSGGGGSGGGGGS (SEQ ID NO: 49). In at least one embodiment, an scFv antibody of the present disclosure can comprise an amino acid sequence of at least 90% sequence identity to a sequence selected from SEQ ID NO: 45 and 46

Although the polypeptide of SEQ ID NO: 49 is exemplified as the scFv antibodies of Table 1, one of ordinary skill in the art will understand a wide range of polypeptide linkers are known in the art and can be used in the scFv antibody and other polypeptide fusion compositions of the present disclosure. Generally, polypeptides comprising polypeptide chains of 5 to 30 amino acids can be used to fuse the polypeptide components of the scFv antibodies and bispecific antibody structures of the present disclosure.

The present disclosure also provides bispecific antibodies, capable of specifically binding to canine, feline and/or equine IL23 and TNFα. In at least one embodiment, the bispecific antibodies comprise the six HVR sequences of the C340 anti-IL23 antibody and the six HVR sequences of the D2E7 anti-TNFα antibody in a single fusion construct. Accordingly, in at least one embodiment the present disclosure provides a “four chain” anti-IL23 LC/HC+anti-TNFα LC/HC bispecific antibody structure that binds to canine, feline, and/or equine IL23 and canine, feline, and/or equine TNFα, wherein the antibody comprises:
(i) an anti-IL23 light chain (LC) comprising a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6);
(ii) an anti-IL23 heavy chain (HC) comprising a VH domain having an HVR-H1 region comprising an amino acid sequence KGSGYSFTTYWLG (SEQ ID NO: 8), an HVR-H2 region comprising an amino acid sequence IMSPVDSDIR (SEQ ID NO: 9), and an HVR-H3 region comprising an amino acid sequence ARRRPGQGYFDF (SEQ ID NO: 10);
(iii) an anti-TNFα light chain (LC) comprising a VL domain an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29); and
(iv) an anti-TNFα heavy chain (HC) comprising a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33).

In some embodiments, the bispecific antibody binds to canine IL23 and canine TNFα and comprises:
(i) the anti-IL23 light chain (LC) comprises a light chain variable domain (VL) amino acid sequence having at least 90% identity to a sequence selected from SEQ ID NO: 3, 11, 13, and 15;
(ii) the anti-IL23 heavy chain (HC) comprises a heavy chain variable domain (VH) amino acid sequence having at least 90% identity to a sequence selected from SEQ ID NO: 7, 12, 14, and 16;
(iii) the anti-TNFα light chain (LC) comprises a light chain variable domain (VL) amino acid sequence having at least 90% identity to a sequence selected from SEQ ID NO: 26, 34, and 36; and
(iv) the anti-TNFα heavy chain (HC) comprises a heavy chain variable domain (VH) amino acid sequence having at least 90% identity to a sequence selected from SEQ ID NO: 30, 35, and 37.

In some embodiments, the present disclosure provides a bispecific antibody that binds to canine IL23 and canine TNFα and comprises:
(i) the anti-IL23 light chain (LC) comprises an amino acid sequence having at least 90% identity to a sequence selected from SEQ ID NO: 19 and 22;
(ii) the anti-IL23 heavy chain (HC) comprises an amino acid sequence having at least 90% sequence identity to SEQ ID NO: 20, 21, and 23;
(iii) the anti-TNFα light chain (LC) comprises an amino acid sequence having at least 90% identity to a sequence selected from SEQ ID NO: 40 and 41; and
(iv) the anti-TNFα heavy chain (HC) comprises an amino acid sequence having at least 90% sequence identity to SEQ ID NO: 42, 43, and 44.

In addition to the anti-IL23 LC/HC+anti-TNFα LC/HC bispecific antibody structure, the present disclosure provides a bispecific antibody comprising an anti-IL23 LC/HC antibody fused to an anti-TNFα scFv antibody structure, or an anti-TNFα LC/HC antibody fused to an anti-IL23 scFv antibody structure. Accordingly, in at least one embodiment, the present disclosure provides a bispecific antibody that binds to canine, feline, and/or equine IL23 and canine, feline, and/or equine TNFα, wherein the antibody comprises:
(i) an anti-IL23 light chain (LC) comprising a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6);
(ii) an anti-IL23 heavy chain (HC) comprising a VH domain having an HVR-H1 region comprising an amino acid sequence KGSGYSFITYWLG (SEQ ID NO: 8), an HVR-H2 region comprising an amino acid sequence IMSPVDSDIR (SEQ ID NO: 9), and an HVR-H3 region comprising an amino acid sequence ARRRPGQGYFDF (SEQ ID NO: 10); and
(iii) an anti-TNFα scFv antibody fused to the HC, wherein the anti-TNFα scFv antibody comprises a VL domain having an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29); and a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33).

In another embodiment, the present disclosure also provides a bispecific antibody that binds to canine, feline, and/or equine IL23 and canine, feline, and/or equine TNFα, wherein the antibody comprises:
(i) an anti-TNFα light chain (LC) comprising a VL domain an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29), and
(ii) an anti-TNFα heavy chain (HC) comprising a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33), and
(iii) an anti-IL23 scFv antibody fused to the HC, wherein the anti-IL23 scFv antibody comprises a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6), and a VH domain having an HVR-H1 region comprising an amino acid sequence KGSGYSFITYWLG (SEQ ID NO: 8), an HVR-H2 region comprising an amino acid sequence IMSPVDSDIR (SEQ ID NO: 9) and an HVR-H3 region comprising an amino acid sequence ARRRPGQGYFDF (SEQ ID NO: 10).

In some embodiments, the antibodies comprise a label or are conjugated to a second moiety. The terms “label” and “detectable label” mean a moiety attached to an antibody or its analyte to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

“Amino acid sequence,” means a sequence of amino acids residues in a peptide or protein. The terms “polypeptide” and “protein” are used interchangeably to refer to a polymer of amino acid residues and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

In at least one embodiment, the anti-IL23 antibody of the present disclosure comprises a light chain variable domain (VL) comprising an HVR-L1 comprising the amino acid sequence of SEQ ID NO: 4, an HVR-L2 comprising the amino acid sequence of SEQ ID NO: 5, and an HVR-L3 comprising the amino acid sequence of SEQ ID NO: 6, and a heavy chain variable domain (VH) comprising an HVR-H1 comprising the amino acid sequence of SEQ ID NO: 8, an HVR-H2 comprising the amino acid sequence of SEQ ID NO: 9, and an HVR-H3 comprising the amino acid sequence of SEQ ID NO: 10.

In at least one embodiment, the anti-IL23 antibody of the present disclosure comprises (i) a light chain variable domain (VL) comprising the amino acid sequence of SEQ ID NO: 3, 11, 13, or 15, or a variant thereof wherein 1, 2, 3, 4, 5, or 6 amino acids of the light chain variable domain (VL) is substituted by a different amino acid; (ii) a heavy chain variable domain (VH) comprising the amino acid sequence of SEQ ID NO: 7, 12, 14, or 16, or a variant thereof wherein 1, 2, 3, 4, 5, or 6 amino acids of the heavy chain variable domain (VH) is substituted by a different amino acid, or (iii) a light chain variable domain (VL) as in (i) and a heavy chain variable domain (VH) as in (ii).

In at least one embodiment, the anti-IL23 antibody of the present disclosure comprises (i) a light chain variable domain (VL) comprising an amino acid sequence having at least 90%, at least 91%, at least 92%, at least 93%, at least 94%, at least 95%, at least 96%, at least 97%, at least 98%, or at least 99% sequence identity to SEQ ID NO: 3, 11, 13, or 15, (ii) a heavy chain variable domain (VH) comprising an amino acid sequence having at least 90%, at least 91%, at least 92%, at least 93%, at least 94%, at least 95%, at least 96%, at least 97%, at least 98%, or at least 99% sequence identity to SEQ ID NO: 7, 12, 14, or 16, or (iii) a light chain variable domain (VL) as in (i) and a heavy chain variable domain (VH) as in (ii).

In at least one embodiment, the anti-TNFα antibody of the present disclosure comprises a light chain variable domain (VL) comprising an HVR-L1 comprising the amino acid sequence of SEQ ID NO: 27, an HVR-L2 comprising the amino acid sequence of SEQ ID NO: 28, and an HVR-L3 comprising the amino acid sequence of SEQ ID NO: 29; and a heavy chain variable domain (VH) comprising an HVR-H1 comprising the amino acid sequence of SEQ ID NO: 31, an HVR-H2 comprising the amino acid sequence of SEQ ID NO: 32, and an HVR-H3 comprising the amino acid sequence of SEQ ID NO: 33.

In at least one embodiment, the anti-TNFα antibody of the present disclosure comprises (i) a light chain variable domain (VL) comprising the amino acid sequence of SEQ ID NO: 26, 34, or 36, or a variant thereof wherein 1, 2, 3, 4, 5, or 6 amino acids of the light chain variable domain (VL) is substituted by a different amino acid; (ii) a heavy chain variable domain (VH) comprising the amino acid sequence of SEQ ID NO: 30, 35, or 37, or a variant thereof wherein 1, 2, 3, 4, 5, or 6 amino acids of the heavy chain variable domain (VH) is substituted by a different amino acid, or (iii) a light chain variable domain (VL) as in (i) and a heavy chain variable domain (VH) as in (ii).

In at least one embodiment, the anti-TNF α antibody comprises (i) a light chain variable domain (VL) comprising an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 26, 34, or 36; (ii) a heavy chain variable domain (VH) comprising an amino acid sequence having at least 90%, at least 91%, at least 92%, at least 93%, at least 94%, at least 95%, at least 96%, at least 97%, at least 98%, or at least 99% sequence identity to the amino acid sequence of SEQ ID NO: 30, 35, or 37; or (iii) a heavy chain variable domain (VH) as in (i) and a heavy chain variable domain (VH) as in (ii).

The term “constant region” or “constant domain” as used herein refers to a region comprising at least three constant domains.

The terms “heavy chain constant region” or “constant heavy chain” are used interchangeably to refer to a region comprising at least three heavy chain constant domains, CH1, CH2, and CH3. Nonlimiting exemplary heavy chain constant regions include γ, δ, α, ε, and μ. Each heavy chain constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising a μ constant region is an IgM antibody, and an antibody comprising an E constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgAQ1 (comprising an α₁ constant region) and IgA2 (comprising an α₂ constant region) antibodies; and IgM antibodies include but are not limited to IgM1 and IgM2.

The terms “light chain constant region” or “constant light chain” are used interchangeably to refer to a region comprising a light chain constant domain, CL. Nonlimiting exemplary light chain constant regions include λ and κ. Non-function-altering deletions and alterations within the domains are encompassed within the scope of the term “constant region” unless designated otherwise. Canine, feline, and equine have antibody classes such as IgG, IgA, IgD, IgE, and IgM. Within the canine IgG antibody class are IgG-A, IgG-B, IgG-C, and IgG-D.

In at least one embodiment, the present disclosure provides a chimeric anti-IL23 antibody comprising: (a) (i) a light chain amino acid sequence of SEQ ID NO: 17; (ii) a heavy chain amino acid sequence of SEQ ID NO: 18; or (iii) a light chain amino acid sequence as in (i) and a heavy chain sequence as in (ii).

In some embodiments, the anti-IL23 and anti-TNF α antibodies of the present disclosure can comprise a canine heavy chain constant region selected from an IgG-A, IgG-B, IgG-C, and IgG-D constant region.

In some embodiments, at least one amino acid residue in a portion of a mouse heavy chain variable domain (VH) or a mouse light chain variable domain (VL) has been replaced with the corresponding amino acid from a canine variable region. In some embodiments, the modified chain is fused to a canine constant heavy chain or a canine constant light chain.

In some embodiments, the caninized TNF α antibody comprises: (i) a light chain VL domain sequence of SEQ ID NO: 11 or 13, or a variant thereof that retains the same HVR sequences and has at least 90%, at least 91%, at least 92%, at least 93%, at least 94%, at least 95%, at least 96%, at least 97%, at least 98%, or at least 99% sequence identity to the amino acid sequence of SEQ ID NO: 11 or 13, (ii) a heavy chain VH domain sequence of SEQ ID NO: 12 or 14 or a variant thereof that retains the same HVR sequences and has at least 90%, at least 91%, at least 92%, at least 93%, at least 94%, at least 95%, at least 96%, at least 97%, at least 98%, or at least 99% sequence identity to the amino acid sequence of SEQ ID NO: 12 or 14, or (iii) a light chain VL domain sequence as in (i) and a heavy chain VL domain sequence as in (ii).

In at least one embodiment, the present disclosure provides a caninized anti-IL23 antibody comprising: (a) (i) a light chain amino acid sequence of SEQ ID NO: 19 or 22; (ii) a heavy chain amino acid sequence of SEQ ID NO: 20, 21, or 23; or (iii) a light chain amino acid sequence as in (i) and a heavy chain sequence as in (ii).

In at least one embodiment, the present disclosure provides a bispecific antibody that binds to canine IL23 and canine TNF α and comprises (i) a light chain variable domain (VL) comprising the amino acid of SEQ ID NO: 19; (ii) a heavy chain variable domain (VH) comprising the amino acid sequence of SEQ ID NO: 47 or 48.

In some embodiments, a biological activity of an Fc polypeptide is the ability to bind FcRn. In some embodiments, a biological activity of an Fc polypeptide is the ability to bind Clq. In some embodiments, a biological activity of an Fc polypeptide is the ability to bind CD16. In some embodiments, a biological activity of an Fc polypeptide is the ability to bind protein A.

In some embodiments, a variant IgG Fc polypeptide comprises a variant IgG Fc polypeptide of a companion animal species. In some embodiments, a variant IgG Fc polypeptide comprises a variant canine IgG Fc polypeptide. In some embodiments, a variant IgG Fc polypeptide (e.g., a variant canine IgG-A Fc polypeptide, a variant canine IgG-C Fc polypeptide, or a variant canine IgG-D Fc polypeptide, variant feline IgG1α Fc polypeptide) has an activity that the reference (e.g., wild-type) polypeptide substantially lacks.

An antibody may be modified to extend or shorten its half-life. In some embodiments involving a higher dose of antibody, a shorter half-life may be desirable for acute treatment. In some embodiments involving a lower dose of antibody, a longer half-life may be desirable for prolonged treatment. For example, as discussed below, mutations in IgG Fc that affect FcRn interactions may be introduced.

In some embodiments, a IL23 and/or TNF α antibody comprises a wild-type or variant IgG Fc having complement fixation activity (or complement-dependent cytotoxicity (CDC)). In some embodiments, a IL23 and/or TNF α antibody comprises a wild-type or variant IgG Fc having antibody-dependent cellular cytotoxicity (ADCC) activity. In some embodiments, a IL23 and/or TNF α antibody comprises a wild-type or variant IgG Fc having antibody-dependent cellular phagocytosis (ADCP) activity. In some embodiments, a IL23 and/or TNF α antibody comprises a wild-type or variant IgG Fc having complement fixation activity and/or ADCC activity and/or ADCP activity. IgG Fc polypeptides may be modified to have an effector function or to have an enhanced effector function.

In some embodiments, a IL23 and/or TNF α antibody comprises a wild-type or variant IgG Fc the binds to canine FcRn at low pH.

In some embodiments, a variant IgG Fc (e.g., a variant canine IgG Fc polypeptide) has modified FcRn binding affinity compared to a reference polypeptide. In some embodiments, a variant IgG Fc has increased FcRn binding affinity at an acidic pH (e.g., at a pH in the range of from about 5.0 to about 6.5, such as at a pH of about 5.0, a pH of about 5.5, a pH of about 6.0, or a pH of about 6.5) compared to a reference polypeptide.

In some embodiments, an anti-IL23 antibody binds to canine IL23, feline IL23, or equine IL23 with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by Biosensor.

In some embodiments, an anti-IL23 antibody binds to canine IL23, human IL23, feline IL23, or equine IL23 with a Kd of between 5×10⁻⁶ M and 1×10⁻⁶ M, between 5×10⁻⁶ M and 5×10⁻⁷ M, between 5×10⁻⁶ M and 1×10⁻⁷ M, between 5×10⁻⁶ M and 5×10⁻⁸ M, 5×10⁻⁶ M and 1×10⁻⁸ M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹ M, between 5×10⁻⁶ M and 1×10⁻¹¹ M, between 5×10⁻⁶ M and 5×10⁻¹² M, between 5×10⁻⁶ M and 1×10⁻¹² M, between 1×10⁻⁶ M and 5×10⁻⁷ M, between 1×10⁻⁶ M and 1×10⁻⁷ M, between 1×10⁻⁶ M and 5×10⁻⁹ M, 1×10⁻⁶ M and 1×10⁻⁸ M, between 1×10⁻⁶ M and 5×10⁻⁹ M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹ M, between 1×10⁻⁶ M and 1×10⁻¹¹ M, between 1×10⁻⁶ M and 5×10⁻¹⁰ M, between 1×10⁻⁶ M and 1×10⁻¹¹ M, between 5×10⁻⁷ M and 1×10⁻⁷ M, between 5×10⁻⁷ M and 5×10⁻⁹ M, 5×10⁻⁷ M and 1×10⁻⁷ M, between 5×10⁻⁷ M and 5×10⁻⁹ M, between 5×10⁻⁷ M and 1×10⁻⁹ M, between 5×10⁻⁹ M and 5×10⁻¹⁰ M, between 5×10⁻⁷ M and 1×10⁻⁹ M, between 5×10⁻⁷ M and 5×10⁻¹¹ M, between 5×10⁻⁷ M and 1×10⁻¹⁰ M, between 5×10⁻⁷ M and 5×10⁻¹² M, between 5×10⁻⁷ M and 1×10⁻¹² M, between 1×10⁻⁷ M and 5×10⁻⁸ M, 1×10⁻⁷ M and 1×10⁻⁸ M, between 1×10⁻⁷ M and 5×10⁻⁹ M, between 1×10⁻⁷ M and 1×10⁻⁹ M, between 1×10⁻⁷ M and 5×10⁻¹⁰ M, between 1×10⁻⁷ M and 1×10⁻¹⁰ M, between 1×10⁻⁷ M and 5×10⁻¹¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹ M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹ M, between 5×10⁻⁸ M and 1×10⁻¹¹ M, between 5×10⁻⁸ M and 5×10⁻¹² M, between 5×10⁻⁸ M and 1×10⁻¹² M, 1×10⁻⁸ M and 5×10⁻⁹ M, between 1×10⁻⁸ M and 1×10⁻⁹ M, between 1×10⁻⁸ M and 5×10⁻¹⁰ M, between 1×10⁻⁸ M and 1×10⁻¹⁰ M, between 1×10⁻⁸ M and 5×10⁻¹¹ M, between 1×10⁻⁸ M and 1×10⁻¹⁰ M, between 1×10⁻⁸ M and 5×10⁻¹⁰ M, between 1×10⁻⁸ M and 1×10⁻¹¹ M, between 5×10⁻⁹ M and 1×10⁻⁹ M, between 5×10⁻⁹ M and 5×10⁻¹⁰ M, between 5×10⁻⁹ M and 1×10⁻¹⁰ M, between 5×10⁻⁹ M and 5×10⁻¹¹ M, between 5×10⁻⁹ M and 1×10⁻¹⁰ M, between 5×10⁻⁹ M and 5×10⁻¹² M, between 5×10⁻⁹ M and 1×10⁻¹¹ M, between 1×10⁻⁹ M and 5×10⁻¹¹ M, between 1×10⁻⁹ M and 1×10⁻¹⁰ M, between 1×10⁻⁹ M and 5×10⁻¹¹ M, between 1×10⁻⁹ M and 1×10⁻¹¹ M, between 1×10⁻⁹ M and 5×10⁻¹² M, between 1×10⁻⁹ M and 1×10⁻¹² M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹ M, 1×10⁻¹⁰ M and 1×10⁻¹¹ M, between 1×10⁻¹⁰ M and 5×10⁻¹² M, between 1×10⁻¹⁰ M and 1×10⁻¹² M, between 5×10⁻¹¹ M and 1×10⁻¹² M, between 5×10⁻¹¹ M and 5×10⁻¹² M, between 5×10⁻¹¹ M and 1×10⁻¹² M, between 1×10⁻¹¹ M and 5×10⁻¹² M, or between 1×10⁻¹¹ M and 1×10⁻¹² M, as measured by biolayer interferometry.

In some embodiments, an anti-IL23 antibody binds to canine IL23, human IL23, feline IL23, or equine IL23, as determined by immunoblot analysis.

In some embodiments, an anti-TNFα antibody binds to canine TNFα, human TNFα, feline TNFα, or equine TNFα with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10−9 M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by a biosensor.

In some embodiments, an anti-TNFα antibody binds to canine TNFα, human TNFα, feline TNFα, or equine TNFα with a Kd of between 5×10⁻⁶ M and 1×10⁻⁶ M, between 5×10⁻⁶ M and 5×10⁻⁷ M, between 5×10⁻⁶ M and 1×10⁻⁷M, between 5×10⁻⁶ M and 5×10⁻⁸ M, 5×10⁻⁶ M and 1×10⁻⁸ M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹ M, between 5×10⁻⁶ M and 1×10⁻¹¹ M, between 5×10⁻⁶ M and 5×10⁻¹² M, between 5×10⁻⁶ M and 1×10⁻¹² M, between 1×10⁻⁶ M and 5×10⁻⁷ M, between 1×10⁻⁶ M and 1×10⁻⁷M, between 1×10⁻⁶ M and 5×10⁻⁸ M, 1×10⁻⁶ M and 1×10⁻⁸ M, between 1×10⁻⁶ M and 5×10⁻⁹ M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹ M, between 1×10⁻⁶ M and 1×10⁻¹¹ M, between 1×10⁻⁶ M and 5×10⁻¹² M, between 1×10⁻⁶ M and 1×10⁻¹² M, between 5×10⁻⁷ M and 1×10⁻⁷ M, between 5×10⁻⁷ M and 5×10⁻⁹ M, 5×10⁻⁷ M and 1×10⁻⁸ M, between 5×10⁻⁷ M and 5×10⁻⁹ M, between 5×10⁻⁷ M and 1×10⁻⁹ M, between 5×10⁻⁷ M and 5×10⁻¹⁰ M, between 5×10⁻⁷ M and 1×10⁻⁸ M, between 5×10⁻⁷ M and 5×10⁻⁹ M, between 5×10⁻⁷ M and 1×10⁻⁹ M, between 5×10⁻⁷M and 5×10⁻¹² M, between 5×10⁻⁷M and 1×10⁻¹² M, between 1×10⁻⁷M and 5×10⁻⁸ M, 1×10⁻⁷ M and 1×10⁻⁸ M, between 1×10⁻⁷ M and 5×10⁻⁹ M, between 1×10⁻⁷ M and 1×10⁻⁹ M, between 1×10⁻⁷ M and 5×10⁻¹⁰ M, between 1×10⁻⁷ M and 1×10⁻¹⁰ M, between 1×10⁻⁷ M and 5×10⁻¹¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹ M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹ M, between 5×10⁻⁸ M and 1×10⁻¹¹ M, between 5×10⁻⁸ M and 5×10⁻¹² M, between 5×10⁻⁸ M and 1×10⁻¹² M, 1×10⁻⁸ M and 5×10⁻⁹ M, between 1×10⁻⁸ M and 1×10⁻⁹ M, between 1×10⁻⁸ M and 5×10⁻¹⁰ M, between 1×10⁻⁸ M and 1×10⁻⁹ M, between 1×10⁻⁸ M and 5×10⁻⁹ M, between 1×10⁻⁸ M and 1×10⁻¹¹ M, between 1×10⁻⁸ M and 5×10⁻¹² M, between 1×10⁻⁸ M and 1×10⁻¹² M, between 5×10⁻⁹ M and 1×10⁻⁹ M, between 5×10⁻⁹ M and 5×10⁻¹⁰ M, between 5×10⁻⁹ M and 1×10⁻¹⁰ M, between 5×10⁻⁹ M and 5×10⁻¹¹ M, between 5×10⁻⁹ M and 1×10⁻¹¹ M, between 5×10⁻⁹ M and 5×10⁻¹² M, between 5×10⁻⁹ M and 1×10⁻¹² M, between 1×10⁻⁹ M and 5×10⁻¹⁰ M, between 1×10⁻⁹ M and 1×10⁻¹⁰ M, between 1×10⁻⁹ M and 5×10⁻¹¹ M, between 1×10⁻⁹ M and 1×10⁻¹¹ M, between 1×10⁻⁹ M and 5×10⁻¹² M, between 1×10⁻⁹M and 1×10⁻¹² M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹ M, 1×10⁻¹⁰ M and 1×10⁻¹¹ M, between 1×10⁻¹⁰ M and 5×10⁻¹² M, between 1×10⁻¹⁰ M and 1×10⁻¹² M, between 5×10⁻¹¹ M and 1×10⁻¹² M, between 5×10⁻¹¹ M and 5×10⁻¹² M, between 5×10⁻¹¹ M and 1×10⁻¹² M, between 1×10⁻¹¹ M and 5×10⁻¹² M, or between 1×10⁻¹¹ M and 1×10⁻¹² M, as measured by a biosensor.

In some embodiments, an anti-TNFα antibody binds to canine TNFα, human TNFα, feline TNFα, or equine TNFα, as determined by immunoblot analysis.

In some embodiments, a variant has at least 1, 2, 3, 4, 5, or 6 amino acids substituted by a different amino acid.

In some embodiments, a variant has at least about 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 97% sequence identity, at least about 98% sequence identity, or at least about 99% sequence identity with the sequence of the reference nucleic acid or polypeptide.

In some embodiments, an anti-IL23 antibody may reduce IL23 signaling function in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to IL23 signaling function in the absence of the antibody. In some embodiments, the reduction in IL23 signaling function is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

In some embodiments, an anti-TNFα antibody may reduce TNFα signaling function in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to NGF signaling function in the absence of the antibody. In some embodiments, the reduction in NGF signaling function is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

### Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water, salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog. In other embodiments, particularly where the antibody is highly stable to thermal and oxidative denaturation, the pharmaceutical composition can be stored as a liquid, i.e., as an aqueous composition, which may be administered directly, or with appropriate dilution, to the dog. A lyophilized composition can be reconstituted with sterile Water for Injection (WFI). Bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2-micron membranes). Sterility may be maintained with or without anti-bacterial agents.

### Exemplary Uses of Antibodies and Pharmaceutical Compositions

The antibodies or pharmaceutical compositions comprising the antibodies of the invention may be useful for treating an IL23 mediated condition, disorder, or disease, in a subject, wherein the subject may be in a companion animal, including, but not limited to, a canine or a feline.

In some embodiments, an anti-IL23 antibody with or without an anti-TNFα antibody or pharmaceutical compositions comprising the same can be utilized in accordance with the methods herein to treat conditions associated with IL23. In some embodiments, an anti-IL23 and/or an anti-TNFα antibody or pharmaceutical composition is administered to a companion animal, such as a canine or a feline, to treat a condition associated with IL23 or TNFα. In some embodiments, an anti-IL23 and/or an anti-TNFα antibody or pharmaceutical composition is administered to a companion animal, such as a canine or a feline, to maintain remission of a condition associated with IL23 and/or TNFα.

The therapeutically effective amount can be an amount one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, an anti-IL23 and/or an anti-TNFα antibody or pharmaceutical composition comprising a IL23 and/or TNFα antibody is administered parenterally, by subcutaneous administration, intravenous infusion, or intramuscular injection. In some embodiments, an anti-IL23 and/or an anti-TNFα antibody or pharmaceutical composition comprising an anti-IL23 and/or an anti-TNFα antibody is administered as a bolus injection or by continuous infusion over a period of time. In some embodiments, an anti-IL23 and/or an anti-TNFα antibody or pharmaceutical composition comprising an anti-IL23 and/or an anti-TNFα antibody is administered by an intramuscular, an intraperitoneal, an intracerebrospinal, a subcutaneous, an intra-arterial, an intrasynovial, an intrathecal, or an inhalation route.

An anti-IL23 antibody alone, combined with an anti-TNFα antibody, or with an TNFα binding region in a bispecific antibody, as described herein, may be administered in an amount in the range of 0.01 mg/kg body weight to 100 mg/kg body weight per dose. In some embodiments, IL23 alone or combined with TNFα antibodies may be administered in an amount in the range of 0.5 mg/kg body weight to 50 mg/kg body weight per dose. In some embodiments, IL23 and/or TNFα antibodies may be administered in an amount in the range of 0.1 mg/kg body weight to 10 mg/kg body weight per dose. In some embodiments, IL23 and/or TNFα antibodies may be administered in an amount in the range of 0.1 mg/kg body weight to 100 mg/kg body weight per dose. In some embodiments, IL23 and/or TNFα antibodies may be administered in an amount in the range of 1 mg/kg body weight to 10 mg/kg body weight per dose. In some embodiments, IL23 and/or TNFα antibodies may be administered in an amount in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, in the range of 0.01 mg/kg body weight to 0.5 mg/kg body weight, in the range of 0.01 mg/kg body weight to 0.1 mg/kg body weight, or in the range of 5 mg/kg body weight to 50 mg/kg body weight. In some embodiments, IL23 and/or TNFα antibodies may be administered in an amount of 0.5 mg/kg body weight. In some embodiments, IL23 and/or TNFα antibodies may be administered in an amount of 2 mg/kg body weight.

A IL23 alone or combined with TNFα antibody or a pharmaceutical composition comprising a IL23 and/or TNFα antibody can be administered to a companion animal at one time or over a series of treatments. For example, a IL23 and/or TNFα antibody or a pharmaceutical composition comprising a IL23 and/or TNFα antibody may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration can be “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the method comprises administering in combination with a IL23 and/or TNFα antibody or a pharmaceutical composition comprising a IL23 and/or TNFα antibody, an IL17 antibody, an IL-5 antibody, an IL-10 antibody, an IL-31 antibody, an IL4 antibody, an IL13 antibody, an IgE antibody, a CD11α antibody, an IL6 antibody, an IL6R antibody, an α4-Intergrin antibody, an beta7-Intergrin antibody, an IL12 antibody, an IL1β antibody, or an anti-BlyS antibody.

Provided herein are methods of exposing to a cell a IL23 and/or TNFα antibody or a pharmaceutical composition comprising a IL23 and/or TNFα antibody under conditions permissive for binding of the antibody to IL23 and/or TNFα. In some embodiments, the cell is exposed to the antibody or pharmaceutical composition ex vivo. In some embodiments, the cell is exposed to the antibody or pharmaceutical composition in vivo. In some embodiments, a cell is exposed to the IL23 and/or TNFα antibody or the pharmaceutical composition under conditions permissive for binding of the antibody to extracellular IL23 and/or TNFα.

In some embodiments, a cell may be exposed in vivo to the IL23 and/or TNFα antibody or the pharmaceutical composition by any one or more of the administration methods described herein, including but not limited to, intraperitoneal, intramuscular, intravenous injection into the subject. In some embodiments, a cell may be exposed ex vivo to the IL23 and/or TNFα antibody or the pharmaceutical composition by exposing the cell to a culture medium comprising the antibody or the pharmaceutical composition. In some embodiments, the permeability of the cell membrane may be affected by the use of any number of methods understood by those of skill in the art (such as electroporating the cells or exposing the cells to a solution containing calcium chloride) before exposing the cell to a culture medium comprising the antibody or the pharmaceutical composition.

In some embodiments, the binding results in a reduction of IL23 and/or TNFα or IL12 signaling function by the cell. In some embodiments, a IL23 and/or TNFα antibody may reduce IL23 and/or TNFα or IL12 signaling function in a cell by at least 10%, at least 15%, at least 20%, at least 25%, at least 30%, at least 35%, at least 40%, at least 45%, at least 50%, at least 60%, at least 70%, at least 80%, at least 90%, or 100% compared to IL23 and/or TN or IL12 signaling function in the absence of the antibody. In some embodiments, the reduction in IL23 and/or TNFα or IL12 signaling function is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Provided herein are methods of using the IL23 and/or TNFα antibodies, polypeptides and polynucleotides for detection, diagnosis and monitoring of a condition associated with IL23 and/or TNFα. Provided herein are methods of determining whether a companion animal will respond to IL23 and/or TNFα antibody therapy. In some embodiments, the method comprises detecting whether the animal has cells that express IL23 and/or TNFα using a IL23 and/or TNFα antibody. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the antibodies or polypeptides described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

In some embodiments, the cells or cell/tissue lysate are contacted with a IL23 and/or TNFα antibody and the binding between the antibody and the cell is determined. When the test cells show binding activity as compared to a reference cell of the same tissue type, it may indicate that the subject would benefit from treatment with a IL23 and/or TNFα antibody. In some embodiments, the test cells are from tissue of a companion animal.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (for example ¹²⁵I, ¹³¹I, ³⁵S, ³H, or ³²P), enzymes (for example, alkaline phosphatase, horseradish peroxidase, luciferase, or p-galactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art. In some embodiments, the IL23 and/or TNFα antibodies need not be labeled, and the presence thereof can be detected using a second labeled antibody which binds to the first IL23 and/or TNFα antibody. In some embodiments, the IL23 and/or TNFα antibody can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The IL23 and/or TNFα antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, ³H, or any other radionuclide label, including those outlined herein) so that the cells or tissue of interest can be localized using immunoscintiography. The antibody may also be used as staining reagent in pathology using techniques well known in the art.

In some embodiments, a first antibody is used for a diagnostic and a second antibody is used as a therapeutic. In some embodiments, the first and second antibodies are different. In some embodiments, the first and second antibodies can both bind to the antigen at the same time, by binding to separate epitopes.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Recombinant Expression of Canine IL23 or p40

A heterodimeric canine IL23 polypeptide sequence for recombinant expression was designed as SEQ ID NO: 1. This canine IL23 polypeptide (SEQ ID NO: 1) includes a linker between the two subunits to stabilize the complex, a polyHis tag for purification, and a tag for in vitro site specific biotinylation. This single chain canine IL23 polypeptide was expressed using in CHO cells as a monomeric protein. The expressed protein was purified using a Ni-NTA column.

A heterodimeric feline IL23 polypeptide sequence for recombinant expression also was designed as SEQ ID NO: 2. This feline IL23 polypeptide (SEQ ID NO: 2) contains a linker between the two subunits to stabilize the complex. A canine p40 polypeptide was also expressed in mammalian CHO cells.

### Example 2: Anti-IL23 Antibody Gene Synthesis, Expression from CHO Cells, and Purification

DNA sequences encoding the anti-IL23 (clone C340) VL domain of SEQ ID NO: 3, and VH domain of SEQ ID NO: 7 were fused to DNA sequences encoding the canine constant CL kappa and IgGB regions. These anti-IL23 (clone C340) genes were synthesized chemically and inserted into an expression vector suitable for transfection into a CHO host cell. These expression vector(s) were transfected into a CHO cell. The chimeric anti-IL23 antibody was expressed in the CHO cells, and the expressed antibody was purified by one or more of various steps including Protein A column chromatography.

Results: The chimeric antibody was well expressed and presented as monodispersed peak on a gel filtration chromatography.

### Example 3: Demonstration of Canine IL23 Binding Activity

Most monoclonal anti-IL23 antibodies failed to bind to non-primate cognate targets. Surprisingly, as shown by the results of the study in this example, it was found that the chimeric anti-IL23 antibodies described herein (e.g., anti-IL23 antibodies of Table 1 above) have high binding affinity to both canine IL23 and canine p40. This study was performed to measure the binding affinity of anti-IL23 antibodies to IL23 and p40 antigens using a Biacore 8K.

### Materials

TABLE 2
Samples used

| Samples | MW(KDa) | Concentration(mg/ml) |
|---|---|---|
| Anti-IL23 (full | 150 | 0.836 |
| length IgG form) | | |
| Canine IL23 | 58 | 1.49 |
| Canine p40 | 38 | 0.68 |

TABLE 3
Instrument and reagents used

| Names | Cat. No. | Lot. No. | Vendor |
|---|---|---|---|
| Biacore 8K | N/A | N/A | Cytiva |
| Series S Sensor | 29-1275-55 | 10319450 | Cytiva |
| Chip Protein A | | | |
| HBS-EP+ buffer | BR-1006-69 | 33049 | Cytiva |
| Regeneration buffer: | | | |
| 10 mM Glycine-HCl pH 1.5 | | | |

### Methods

### 1. Affinity Measurement of Antigens to Antibodies

Preparation of running buffer: Dilute one volume of the 10× buffer with 9 volumes of degassed, filtered MilliQ water.

Preparation of regeneration buffer (10 mM glycine): Weigh a certain amount of glycine and dissolve it in MilliQ water. Adjust the pH to 1.5~1.7 to obtain regeneration buffer.

The assay was performed at 25° C. and the running buffer was HBS-EP+.

Antibodies were injected as capture on the Series S Sensor Chip Protein A.

Antigens were diluted into multiple concentrations and injected over the surface of flow cell 1 and 2 as association phase, followed by injecting running buffer as dissociation phase.

The binding model was showed below. Running configuration used is listed in Table 4 below.

TABLE 4
Running configuration
Capture

| Ligand | antibodies |
|---|---|
| Capture Time(s) | 30 |

Association & Dissociation

| | |
|---|---|
| Association contact time(s) | 120 |
| Dissociation contact time(s) | 360 |
| Flow rate(μl/min) | 30 |
| Sample concentrations(nM) | 12.5, 6.25, 3.125, 1.5625, |
| | 0.78125, 0.390625 (Canine |
| | IL23/canine p40 to |
| | Anti-IL23-VH-VL) |

Surface Regeneration

| | |
|---|---|
| Contact time(s) | 30 |
| Flow rate(μl/min) | 30 |
| Regeneration buffer: | 10 mM Glycine-HCl pH 1.5 |

### Results

All the data were processed using the Biacore 8K Evaluation software version 3.0. Flow cell 1 and blank injection of buffer in each cycle were used as double reference for Response Units subtraction. The Biacore binding affinity values are provided in Table 5 below.

TABLE 5
Affinity measurement of antibodies to antigens

| Ligand | Analyte | Chi² (RU²) | ka (1/Ms) | kd (1/s) | KD (M) | Rmax (RU) |
|---|---|---|---|---|---|---|
| Anti-IL23-VH-VL | Canine IL23 | 9.86E−02 | 5.83E+06 | 3.38E−03 | 5.80E−10 | 25.7 |
| Anti-IL23-VH-VL | p40 | 2.34E−01 | 4.68E+06 | 4.78E−03 | 1.02E−09 | 38.4 |

### Example 4: Caninization of the Anti-H123 C340 Antibody

This example illustrates a study of the caninization of the anti-IL23 C340 antibody having the VL domain of SEQ ID NO: 3 and the VH domain of SEQ ID NO: 7. An initial analysis of the anti-IL23 i40 VL and VH amino acid sequences identified candidate amino acid substitutions at various positions that could be used to caninize the sequences. A listing of these candidate amino acid substitutions is provided in Table 6 below.

TABLE 6
Candidate VL (SEQ ID NO: 3) and VH (SEQ
ID NO: 7) amino acid substitutions

| | VL Domain | | VH Domain |
|---|---|---|---|
| | Candidate | | Candidate |
| VL Domain | Caninization | VH Domain | Caninization |
| (SEQ ID NO: 3) | Amino Acid | (SEQ ID NO: 7) | Amino Acid |
| Amino Acids | Substitutions | Amino Acids | Substitutions |
| D | Q | E | D |
| I | S | V | |
| Q | V, L | Q | |
| M | L, V | L | |
| T | | V | |
| Q | | Q | E |
| S | T, P, L, E | S | |
| P | | G | A |
| S | L, A, T, | A | G |
| S | | E | D |
| L | V | V | L |
| S | A | K | V |
| A | V, G | K | |
| S | A | P | |
| V | P, L | G | A |
| G | | E | G, A |
| D | E, Q | S | |
| R | P, T | L | V |
| V | A | K | R |
| T | S | I | L, V |
| I | | S | |
| T | S | C | |
| C | | K | V |
| R | K, T, S | G | A, T |
| A | S, G | S | |
| S | G | G | |
| Q | | Y | |
| G | | S | |
| I | | F | |
| S | | T | |
| S | | T | |
| W | | Y | |
| L | | W | |
| A | | L | |
| W | | G | |
| Y | F | W | |
| Q | L, R, K | V | I |
| Q | H | R | Q |
| K | R, L, F, V | Q | R |
| P | A | M | A |
| E | G | P | |
| K | Q, R, K | G | |
| A | S, G | K | A |
| P | | G | |
| K | R, Q, S | L | |
| S | L, R, T, F | D | Q |
| L | I | W | |
| I | P, V | I | V, M |
| Y | E, Q, N, T, H | G | A |
| A | | I | |
| A | | M | |
| S | | S | |
| S | | P | |
| L | | V | |
| Q | | D | |
| S | | S | |
| G | | D | |
| V | | I | |
| P | | R | |
| S | D, E, V | Y | |
| R | | S | A, T |
| F | | P | D, Q, N |
| S | | S | A, K, V |
| G | | F | V |
| S | | Q | K |
| G | R, N, K, S | G | |
| S | | Q | R |
| G | | V | F, I |
| T | N, S, D | T | |
| D | T, S | M | L, I, F |
| f | A | S | T |
| T | | V | R, A |
| L | | D | |
| T | R, S | K | N, T |
| I | | S | A, P |
| S | T, G | I | K, T, R |
| S | R, G | T | N, G |
| L | V, I | T | |
| Q | E | A | L, V, T, I |
| P | A | Y | |
| E | D | L | M |
| D | | Q | E, |
| F | A, T, E | W | M, L |
| A | G | N | S |
| T | V, I, D, A | S | |
| Y | | L | |
| Y | | K | R |
| C | | A | V. S |
| Q | | S | E, G |
| Q | | D | |
| Y | | T | |
| N | | A | G, V |
| I | | M | V |
| Y | | Y | |
| P | | Y | |
| Y | | C | |
| T | | A | |
| F | | R | |
| G | | R | |
| Q | | R | |
| G | | P | |
| T | | G | |
| K | | Q | |
| L | | G | |
| E | | Y | |
| I | | F | |
| K | | D | |
| | | F | |
| | | W | |
| | | G | |
| | | Q | |
| | | G | |
| | | T | |
| | | L | |
| | | V | |
| | | T | |
| | | V | |
| | | S | |
| | | S | |

Based on the candidate amino acid substitutions, genes encoding a number of caninized variants of VL (SEQ ID NO: 3) and VH (SEQ ID NO: 7) and expressed using a human IgG1 and kappa framework. The amino acid sequences of the caninized variants of VL and VH are aligned and listed in Tables 7 and 8 below (the “-” sign indicates positions of amino acid changes).

TABLE 7
Alignment of Caninized VL domains
based on SEQ ID NO: 3

| | |
|---|---|
| C340 | DIQMTQSPSS LSASVGDRVT ITCRASQGIS SWLAWYQQKP |
| V1 | EIVTQSPSAS LSALVQEEKT ITCRASQGIS SWLAWYQQKP |
| V2 | DIVMTQTPLS LSVSPGEPAS ISCRASQGIS SWLAWYLQKA |
| V3 | EIVTQSPSAS LSALVQEEKT ITCRASQGIS SWLAWYQQKP |
| V4 | EIVTQSPSAS LSALVQEEKT ITCRASQGIS SWLAWYQQKP |
| V2.1 | DIVMTQTPLS LSVSPGEPAS ISCRASQGIS SWLAWYQQKA |
| | - ------- -------- - - - |
| | EKAPKSLIYA ASSLQSGVPS RFSGSGSGTD FTLTISSLQP |
| | GQAPKLLIYA ASSLQSGVPS RFSGSGSGTD FTFTISSLEP |
| | GQSPRLLPYA ASSLQSGVPD RFSGSGSGTD FTLRIGRVEA |
| | GQAPKSLIYA ASSLQSGVPS RFSGSGSGTD FTFTISSLEP |
| | EKAPKSLIYA ASSLQSGVPS RFSGSGSGTD FTFTISSLEP |
| | GQAPKSLIYA ASSLQSGVPS RFSGSGSGTD FTFTISSLEP |
| | EKAPKSLIYA ASSLQSGVPD RFSGSGSGTD FTLRIGRVEA |
| | --- -- - -- ----- |
| | EDFATYYCQQ YNIYPYTFGQ GTKLEIK |
| | EDVAVYYCQQ YNIYPYTFGQ GTKLEIK |
| | EDAGIYYCQQ YNIYPYTFGQ GTRLEVR |
| | EDVAVYYCQQ YNIYPYTFGQ GTKLEIK |
| | EDVAVYYCQQ YNIYPYTFGQ GTKLEIK |
| | EDVAVYYCQQ YNIYPYTFGQ GTKLEIK |
| | EDAGIYYCQQ YNIYPYTFGQ GTRLEVR |
| | --- - -- |

TABLE 8
Alignment of Caninized VH domains based
on SEQ ID NO: 7

| | |
|---|---|
| C340 | EVQLVQSGAE VKKPGESLKI SCKGSGYSFT TYWLGWVRQM |
| V1 | EVOLVQSGAE VKKPGASVKV SCKGSGYSFT TYWLGWVRQA |
| V2 | EVQVVESGGD LVKPGGSLKL SCKGSGYSFT TYWLGWVRQA |
| V3 | EVQLVQSAAE VKKPGASVKV SCKGSGYSFT TYWLGWIQRA |
| V4 | EVQLVQSGAE VKKPGASVKV SCKGSGYSFT TYWLGWVRQA |
| V5 | EVQLVQSGAE VKKPGASVKV SCKGSGYSFT TYWLGWVRQM |
| V6 | EVOLVESGGD LVKPGGSLRL SCKGSGYSFT TYWLGWVRQA |
| V1.1 | EVQLVQSGAE VKKPGASVKV SCKGSGYSFT TYWLGWVRQA |
| | --- --- -- - --- --- |
| | PGKGLDWIGI MSPVDSDIRY SPSFQGQVTM SVDKSITTAY |
| | PGAGLDWMGI MSPVDSDIRY AGKFQGQVTM SVDTSTSTAY |
| | PGKGLQWIAI MSPVDSDIRY ANVVKGRITI SRDNPKNTIY |
| | PGAGLDWMGI MSPVDSDIRH AQKFQGRVTL TADTSTGTTY |
| | PGAGLDWMGI MSPVDSDIRY SPSFQGQVTM SVDTSTSTAY |
| | PGKGLDWIGI MSPVDSDIRY SPSFQGQVTM SVDTSTSTAY |
| | PGKGLQWIGI MSPVDSDIRY ADAVKGRFTV SRDNAKRTLY |
| | PGAGLDWMGI MSPVDSDIRY SPSFQGQVTM SVDTSTSTAY |
| | - - -- ----- -- - -- ---- - |
| | LQWNSLKASD TAMYYCARRR PGQGYFDFWG QGTLVTVSS |
| | MELSSLRAGD IAVYYCARRR PGQGYFDFWG QGTLVTVSS |
| | LEMNSLSVED TVMYYCARRR PGQGYFDFWG QGTLVTVSS |
| | TELSSLRAED TAVYYCARRR PGQGYFDFWG QGTQVIVSA |
| | MELSSLRAGD IAVYYCARRR PGQGYFDFWG QGTLVTVSS |
| | MELSSLRAGD IAVYYCARRR PGQGYFDFWG QGTLVTVSS |
| | LQMNSLRAED TAIYYCARRR_PGQGYFDFWG QGTLVTVSS |
| | MELSSLRAGD IAVYYCARRR PGQGYFDFWG QGTLVTVSS |
| | ---- --- --- - - - |

Genes encoding each of the caninized VL and VH variants listed in Tables 7 and 8 above were expressed in a mammalian cell system. Various combinations of anti-IL23 VL and VH polypeptides were tested for the expression and canine IL23 binding.

As shown by the results listed in Table 9 (below), the “wild-type” anti-IL23 C340 VL and VH domain polypeptides were expressed well. Surprisingly, some versions of caninized VH domain polypeptides (e.g., variant V1) were found to pair well with the “wild-type” C340 VL (SEQ ID NO: 3). Similarly, caninized VL (e.g., variant V2) pairs well with the wild-type C340 VH (SEQ ID NO: 7). But the caninized VH domain variant V1 failed to pair with the caninized VL domain variant V2. Further results of these studies are provided in Table 9.

TABLE 9
Description of caninization expression

| VL | VH | Expression¹/Binding² |
|---|---|---|
| C340 | C340 | Express well/Active |
| C340 | V1 | Express well/Active |
| C340 | V2 | Low expression |
| C340 | V3 | Low expression |
| V1 | C340 | Low expression |
| V1 | V1 | Low expression |
| V1 | V2 | Low expression |
| V1 | V3 | Low expression |
| V2 | V1 | Low expression |
| V2 | V2 | Low expression |
| V2 | C340 | Express well/Active |
| V2 | V3 | Low expression |
| C340 | V4 | Express well |
| C340 | V5 | Express well |
| V3 | C340 | Low expression |
| V4 | C340 | Low expression |
| V3 | V4 | Low expression |
| V3 | V5 | Low expression |
| V4 | V4 | Low expression |
| V4 | V5 | Low expression |
| V2 | V4 | Low expression |
| V2 | V5 | Low expression |
| V2 | V6 | Express medium/low Activity |
| V5 | V4 | Low expression |
| V5 | V5 | Low expression |
| V5 | V6 | Express well/low Activity |
| V2.1 | V1.1 | Express well/Active |

¹“Express well” indicates >50 μg/mL; “Low expression” indicates <50 μg/mL to not detectable. Measured using OD at 280 nm.
²“Active” indicates specific binding to canine IL23 as measured using Biacore affinity assay.

As an exemplary caninized anti-IL23, the variants comprising VL domain V2.1 (SEQ ID NO: 11) and VH domain V1.1 (SEQ ID NO: 12) were expressed well and observed to maintain full canine IL23 binding activity using Biacore binding affinity assay.

A mutation was also introduced into the Fc region at the amino acid position with EU numbering 252 located in the AB-turn. This mutation changes position 252 amino acid to Y (L252Y) is described in U.S. Pat. No. 7,658,921 B2, and results in extending the in vivo half-life of IgG antibodies. This half-life extending Fc region mutation was introduced into caninized anti-IL23 heavy chain IgG-B as L252Y resulting in the “long acting” caninized anti-IL23 of SEQ ID NO: 20.

### Example 5: Felinization of the Anti-IL23 (C340) Antibody

This example illustrates a study of the felinization of the anti-IL23 (C340) having the VL domain of SEQ ID NO: 3 and the VH domain of SEQ ID NO: 7.

The felinization of the VL and VH domains is carried using the same general methodology described for caninization in Example 4. An initial analysis of the anti-IL23 C340 VL and VH amino acid sequences is carried out to identify candidate amino acid substitutions at various positions that could be used to felinize the sequences. Genes encoding felinized variants of VL (SEQ ID NO: 3) and VH (SEQ ID NO: 7) using the feline IgG framework are expressed in a mammalian cell system. Feline IL23 binding activity of the expressed felinized variants is measured to identify. Various combinations of anti-IL23 VL and VH polypeptides were tested for the expression and canine IL23 binding

The felinized anti-IL23 antibodies can also be expressed using a half-life extension Fc by replacing “Y” in feline Fc at EU numbering 252 amino acid position. This half-life extending Fc region mutation is introduced into felinized anti-IL23 heavy chain IgG-B as L252Y resulting in the “long acting” felinized anti-IL23.

### Example 6: Caninization of the Anti-TNFα D2E7 Antibody

This example illustrates a study of the caninization of the anti-TNFα D2E7 having the VL domain of SEQ ID NO: 26 and the VH domain of SEQ ID NO: 30.

The resulting caninized anti-TNFα D2E7 VL domain sequence of SEQ ID NO: 34 and VH domain sequence of SEQ ID NO: 35 were used to generate caninized anti-TNFα D2E7 antibodies. A caninized anti-TNFα D2E7 antibody was generated comprising a caninized VL domain of SEQ ID NO: 34 linked to a canine kappa constant region to provide the light chain sequence of SEQ ID NO: 40, and a caninized VH domain of SEQ ID NO: 35 linked to the canine IgG-B constant region to provide the heavy chain sequence of SEQ ID NO: 41. This caninized anti-TNFα was expressed using mammalian Expi293F cells.

This example demonstrates that caninized anti-TNFα antibodies described herein, have high binding affinity to canine TNFα (Sino Biologicals). This study was performed to measure the binding affinity of antibodies to antigens using a Biacore 8K.

### Material

TABLE 10
Samples used

| Samples | MW(KDa) | Concentration(mg/ml) |
|---|---|---|
| Caninized Anti-TNF-VH-VL | 150 | 1.587 |
| Canine TNF-alpha | 17.3 | 1 |

TABLE 11
Instrument and reagents used

| Names | Cat. No. | Lot. No. | Vendor |
|---|---|---|---|
| Biacore 8K | N/A | N/A | Cytiva |
| Series S Sensor Chip Protein A | 29-1275-55 | 10319450 | Cytiva |
| HBS-EP+ buffer | BR-1006-69 | 33049 | Cytiva |
| Regeneration buffer: | | | |
| 10 mM Glycine-HCl pH 1.5 | | | |

### Methods

### 1. Affinity Measurement of Antigens to Antibodies

Preparation of running buffer: Dilute one volume of the 1 Ox buffer with 9 volumes of degassed, filtered MilliQ water.

Preparation of regeneration buffer (10 mM glycine): Weigh a certain amount of glycine and dissolve it in MilliQ water. Adjust the pH to 1.5~1.7 to obtain regeneration buffer.

The assay was performed at 25° C. and the running buffer was HBS-EP+.

Antibodies were injected as capture on the Series S Sensor Chip Protein A.

Antigens were diluted into multiple concentrations and injected over the surface of flow cell 1 and 2 as association phase, followed by injecting running buffer as dissociation phase.

The binding model was showed below. Running configuration used is listed in Table 12 below.

TABLE 12
Running configuration
Capture

| Ligand | antibodies |
|---|---|
| Capture Time(s) | 30 |

Association & Dissociation

| | |
|---|---|
| Association contact time(s) | 120 |
| Dissociation contact time(s) | 360 |
| Flow rate(μl/min) | 30 |
| Sample concentrations(nM) | 200, 100, 50, 25, 12.5, 6.25 |
| | (canine TNF-alpha to caninized |
| | Anti-TNF-VH-VL) |

Surface Regeneration

| | |
|---|---|
| Contact time(s) | 30 |
| Flow rate(μl/min) | 30 |
| Regeneration buffer: | 10 mM Glycine-HCl |
| | pH 1.5 |

### Results

All the data were processed using the Biacore 8K Evaluation software version 3.0. Flow cell 1 and blank injection of buffer in each cycle were used as double reference for Response Units subtraction. The Biacore binding affinity values are provided in Table 13 below.

TABLE 13
Affinity measurement of antibodies to antigens

| Ligand | Analyte | Chi² (RU²) | ka (1/Ms) | kd (1/s) | KD (M) | Rmax (RU) |
|---|---|---|---|---|---|---|
| Caninized Anti-TNF-VH- | Canine TNF- | 1.10E−01 | 2.09E+05 | 8.18E−04 | 3.91E−09 | 23.5 |
| VL | alpha | | | | | |

The caninized version of the anti-TNFα D2E7 antibody comprising a light chain (LC) of SEQ ID NO: 40, which has a caninized variable domain (VL) domain linked to the canine kappa constant region, and a heavy chain of SEQ ID NO: 43, which has a caninized variable domain (VH) linked to a canine IgG-B constant region, which further includes a Fc region with a “long-acting” 252Y variant in the Fc at EU numbering). A caninized anti-TNFα has also been designed with long-acting Fc variants in the IgG-A, IgG-C, and IgG-D constant regions.

A felinized version of the anti-TNFα D2E7 antibody has also been designed comprising a felinized light chain variable domain (VL) of D2E7 (SEQ ID NO: 36) feline kappa and a felinized heavy chain variable domain (VH) of D2E7 (SEQ ID NO: 37), and feline IgGa, or IgGb where the canine IgG's Fc may be modified by replacing “Y” in the Fc at EU numbering 252 amino acid position.

### Example 7: Expression and Purification of a Bispecific Anti-Canine IL23 and Anti-Canine TNFα Molecule from CHO Cells

The caninized versions of the anti-TNFα VH and VL domains provided in Table 1 may be used to form an scFv antibody. The sequence structure of two exemplary scFv anti-TNFα antibodies is illustrated in Table 1 by the amino acid sequences of SEQ ID NO: 45 or 46. The scFv antibody sequence of SEQ ID NO: 46 also includes a double Cys variant that allows for disulfide bond formation.

Using anti-TNFα scFv molecules, and the caninized anti-IL23 antibodies described in Table 1, a first exemplary bispecific fusion molecule that specifically binds to both canine IL23 and canine TNFα was designed with the following fusion structure: (1) the caninized anti-IL23 light chain of SEQ ID NO: 19, which includes a caninized variable domain (VL) linked to a canine kappa light constant region; (2) the caninized anti-IL23 heavy chain of SEQ ID NO: 20, which includes a caninized variable domain (VH) linked to a canine IgG-B constant region with long-acting 252Y Fc region; and (3) an scFv caninized anti-TNFα antibody SEQ ID NO: 45. The complete bispecific anti-IL23/anti-TNFα antibody fusion structures are provided in Table 1 as the amino acid sequences of SEQ ID NO: 47 and 48. The bispecific anti-IL23/anti-TNFα antibody of SEQ ID NO: 48 includes the scFv anti-TNFα antibody of SEQ ID NO: 46, which has two cysteines to allow intramolecular S-S bond formation. The resulting complete bispecific antibody fusion structure with the scFv of SEQ ID NO: 46 is provided as the amino acid sequence of SEQ ID NO: 48. The bispecific antibody molecules of SEQ ID NO: 47 and 48 are expressed from mammalian cells and purified by single step Protein A column chromatography.

A second exemplary bispecific anti-IL23/anti-TNFα antibody that specifically binds to both canine IL23 and canine TNFα was designed with the following four-chain structure formed by combining: (1) the anti-IL23 antibody structure having the caninized VH domain containing light chain of SEQ ID NO: 19 and the caninized VH containing heavy chain of SEQ ID NO: 21, which includes a “knob” in its heavy chain; and (2) the anti-TNFα antibody structure formed having the caninized VH domain containing light chain of SEQ ID NO: 41 and the caninized VH containing heavy chain of SEQ ID NO: 44, which includes a “hole” in its heavy chain. Additionally, as shown by the sequences in Table 1, the anti-TNFα LC of SEQ ID NO: 41 and the anti-TNFα HC of SEQ ID NO; 44 were engineered as follows: (1) in the anti-TNFα light chain of SEQ ID NO: 41, a cysteine is removed from CL region, and a cysteine is added in the VL domain; and (2) in the anti-TNFα heavy chain of SEQ ID NO: 44, a cysteine in the CH1 region is removed, and a cysteine is added into VH domain. After co-transfection into CHO cells, the plasmids encoding the four chains of the bispecific anti-IL23/anti-TNFα antibody was expressed very well and mono-dispersed. Mass spectrometry of the protein obtained deglycosylation and reduction with DTT indicated the expect 1:1:1:1 ratio.

### Example 8: Study for Treatment of Canine IBD with Anti-IL23 Antibodies

The effectiveness and safety of anti IL23 for the management of inflammatory bowel disease (IBD) in dogs may be evaluated using various doses ranging from 0.01 mg/kg to 100 mg/kg.

All dogs receive doses of either caninized IL23 antibody or caninized long-acting antibody, with the first dose administered at Day 0. Subsequent doses are administered every week, biweekly, monthly or 2 months, 3 months, up to half year.

Histopathology of endoscopic gastrointestinal biopsies may be used to determine the effectiveness of caninized IL23 antibody in management of IBD.

IBD biomarkers may be measured including, but not limited to IL-1beta, IL6, IL8, IL9, IFN-gamma, TNFα, CCL2, IL22, CRP, LL37, TFF3, OSM.

Canine Inflammatory Bowel Disease Activity Index (CIBDAI) Score may be used for screening treatment candidate.

Controlled conditions may include diet.

### Example 9: Study for Treatment of Canine IBD with Combination Therapy Using Anti-IL23 and Anti-TNFα Antibodies

The effectiveness and safety of a IL23 monoclonal antibody combined with a TNFα monoclonal antibody for the management of inflammatory bowel disease (IBD) in dogs may be evaluated using various doses IL23 monoclonal antibody ranging from 0.01 mg/kg to 100 mg/kg in any combinations with TNFα monoclonal antibody ranging from 0.01 mg/kg to 100 mg/kg.

All dogs receive doses of either caninized IL23 antibody or caninized IL23 antibody long-acting antibody in combinations either caninized TNFα antibody or caninized IL23 long-acting antibody the first dose administered at Day 0. Subsequent doses are administered every week, biweekly, monthly or 2 months, 3 months, up to half year or more.

Alternatively, the effectiveness and safety of a IL23/TNFα bispecific antibody may be used for treatment or management of inflammatory bowel disease (IBD) in dogs may be evaluated using various doses ranging from 0.01 mg/kg to 100 mg/kg.

Histopathology of endoscopic gastrointestinal biopsies may be used to determine the effectiveness of caninized IL23 antibody in management of IBD.

IBD biomarkers may be measured including, but not limited to IL-1beta, IL6, IL8, IL9, IFN-gamma, TNFα, CCL2, IL22, CRP, LL37, TFF3, OSM.

Canine Inflammatory Bowel Disease Activity Index (CIBDAI) Score may be used for screening treatment candidate.

Controlled conditions may include diet.

What is claimed is:

1. An anti-IL23 antibody that binds to canine, feline, and/or equine IL23 comprising (i) a first light chain hypervariable region (HVR-L1), a second light chain hypervariable region (HVR-L2), and a third light chain hypervariable region (HVR-L3), and/or (ii) a first heavy chain hypervariable region (HVR-H1), a second heavy chain hypervariable region (HVR-H2), and a third heavy chain hypervariable region (HVR-H3), wherein:
(a) the HVR-L1 region comprises amino acid sequence RASQGISSWLA (SEQ ID NO: 4), the HVR-L2 region comprises amino acid sequence YAASSLQS (SEQ ID NO: 5), and the HVR-L3 region comprises amino acid sequence QQYNIYPYT (SEQ ID NO: 6); and/or
(b) the HVR-H1 region comprises amino acid sequence KGSGYSFITYWLG (SEQ ID NO: 8), the HVR-H2 region comprises amino acid sequence IMSPVDSDIR (SEQ ID NO: 9) and the HVR-H3 region comprises amino acid sequence ARRRPGQGYFDF (SEQ ID NO: 10).

2. The antibody of claim 1, wherein the antibody is caninized, felinized, or equinized.

3. The antibody of claim 1, wherein the antibody comprises a light chain variable domain (VL) amino acid sequence having at least 90% identity to a sequence selected from SEQ ID NO: 3, 11, 13, and 15; and/or a heavy chain variable domain (VH) amino acid sequence having at least 90% identity to a sequence selected from SEQ ID NO: 7, 12, 14, and 16; optionally, wherein:
(i) the light chain variable domain (VL) comprises a variant of SEQ ID NO: 3, 11, 13, and 15, wherein from 1 to 6 amino acids of the light chain variable domain (VL) are substituted by a different amino acid; and/or
(ii) the heavy chain variable domain (VH) comprises a variant of SEQ ID NO: 7, 12, 14, and 16, wherein from 1 to 6 amino acids of the heavy chain variable domain (VH) are substituted by a different amino acid.

4. The antibody of claim 1, wherein the antibody comprises a light chain variable domain (VL) comprises an amino acid sequence selected from SEQ ID NO: 3, 11, 13, and 15, and/or a heavy chain variable domain (VH) comprises an amino acid sequence selected from SEQ ID NO: 7, 12, 14, and 16; optionally, wherein:
(i) the antibody comprises a light chain variable domain (VL) comprising an amino acid sequence selected from SEQ ID NO: 3 and a heavy chain variable domain (VH) comprising an amino acid sequence selected from SEQ ID NO: 7;
(ii) the antibody comprises a light chain variable domain (VL) comprising an amino acid sequence selected from SEQ ID NO: 11 and a heavy chain variable domain (VH) comprising an amino acid sequence selected from SEQ ID NO: 12;
(iii) the antibody comprises a light chain variable domain (VL) comprising an amino acid sequence selected from SEQ ID NO: 13 and a heavy chain variable domain (VH) comprising an amino acid sequence selected from SEQ ID NO: 14; or
(iv) the antibody comprises a light chain variable domain (VL) comprising an amino acid sequence selected from SEQ ID NO: 15 and a heavy chain variable domain (VH) comprising an amino acid sequence selected from SEQ ID NO: 16.

5. The antibody of any one of claims 1-4, wherein the antibody comprises: a light chain (LC) amino acid sequence having at least 90% identity to an amino acid sequence selected from SEQ ID NO: 17, 19, 22, and 24, and/or a heavy chain (HC) amino acid sequence having at least 90% identity to SEQ ID NO: 18, 20, 21, 23, and 25; optionally, wherein the antibody comprises:
(i) the LC amino acid sequence of SEQ ID NO: 17, and the HC amino acid sequence of SEQ ID NO: 18;
(ii) the LC amino acid sequence of SEQ ID NO: 19, and the HC amino acid sequence of SEQ ID NO: 20;
(iii) the LC amino acid sequence of SEQ ID NO: 19, and the HC amino acid sequence of SEQ ID NO: 21;
(iv) the LC amino acid sequence of SEQ ID NO: 22, and the HC amino acid sequence of SEQ ID NO: 23; or
(v) the LC amino acid sequence of SEQ ID NO: 24, and the HC amino acid sequence of SEQ ID NO: 25.

6. An anti-TNFα antibody that binds to canine, feline, and/or equine TNFα comprising (i) a first light chain hypervariable region (HVR-L1), a second light chain hypervariable region (HVR-L2), and a third light chain hypervariable region (HVR-L3), and/or (ii) a first heavy chain hypervariable region (HVR-H1), a second heavy chain hypervariable region (HVR-H2), and a third heavy chain hypervariable region (HVR-H3), wherein:
(a) an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29); and/or
(a) an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33).

7. The antibody of claim 6, wherein the antibody is caninized, felinized, or equinized.

8. The antibody of claim 6, wherein the antibody comprises a light chain variable domain (VL) amino acid sequence having at least 90% identity to a sequence selected from SEQ ID NO: 26, 34, and 36; and/or a heavy chain variable domain (VH) amino acid sequence having at least 90% identity to a sequence selected from SEQ ID NO: 30, 35, and 37; optionally, wherein:
(i) the light chain variable domain (VL) comprises a variant of SEQ ID NO: 26, 34, and 36, wherein from 1 to 6 amino acids of the light chain variable domain (VL) are substituted by a different amino acid; and/or
(ii) the heavy chain variable domain (VH) comprises a variant of SEQ ID NO: 30, 35, and 37, wherein from 1 to 6 amino acids of the heavy chain variable domain (VH) are substituted by a different amino acid.

9. The antibody of claim 6, wherein the antibody comprises a light chain variable domain (VL) comprises an amino acid sequence selected from SEQ ID NO: 26, 34, and 36, and/or a heavy chain variable domain (VH) comprises an amino acid sequence selected from SEQ ID NO: 30, 35, and 37; optionally, wherein the antibody comprises:
(i) a light chain variable domain (VL) comprising an amino acid sequence selected from SEQ ID NO: 26 and a heavy chain variable domain (VH) comprising an amino acid sequence selected from SEQ ID NO: 30;
(ii) a light chain variable domain (VL) comprising an amino acid sequence selected from SEQ ID NO: 34 and a heavy chain variable domain (VH) comprising an amino acid sequence selected from SEQ ID NO: 35;
(iii) a light chain variable domain (VL) comprising an amino acid sequence selected from SEQ ID NO: 36 and a heavy chain variable domain (VH) comprising an amino acid sequence selected from SEQ ID NO: 37;
(iv) a light chain variable domain (VL) comprising an amino acid sequence selected from SEQ ID NO: 36 and a heavy chain variable domain (VH) comprising an amino acid sequence selected from SEQ ID NO: 37;
(v) a light chain variable domain (VL) comprising an amino acid sequence selected from SEQ ID NO: 36 and a heavy chain variable domain (VH) comprising an amino acid sequence selected from SEQ ID NO: 30; or
(vi) a light chain variable domain (VL) comprising an amino acid sequence selected from SEQ ID NO: 26 and a heavy chain variable domain (VH) comprising an amino acid sequence selected from SEQ ID NO: 37.

10. The antibody of any one of claims 6-9, wherein the antibody comprises: a light chain (LC) amino acid sequence having at least 90% identity to a sequence selected from SEQ ID NO: 38, 40, and 41 and/or a heavy chain (HC) amino acid sequence having at least 90% identity to a sequence selected from SEQ ID NO: 39, 42, 43, and 44; optionally, wherein the antibody comprises:
(i) the LC amino acid sequence of SEQ ID NO: 38, and the HC amino acid sequence of SEQ ID NO: 39;
(ii) the LC amino acid sequence of SEQ ID NO: 40, and the HC amino acid sequence of SEQ ID NO: 42;
(iii) the LC amino acid sequence of SEQ ID NO: 41, and the HC amino acid sequence of SEQ ID NO: 42;
(iv) the LC amino acid sequence of SEQ ID NO: 40, and the HC amino acid sequence of SEQ ID NO: 43;
(v) the LC amino acid sequence of SEQ ID NO: 41, and the HC amino acid sequence of SEQ ID NO: 43;
(vi) the LC amino acid sequence of SEQ ID NO: 40, and the HC amino acid sequence of SEQ ID NO: 44; or
(vi) the LC amino acid sequence of SEQ ID NO: 41, and the HC amino acid sequence of SEQ ID NO: 44.

11. The antibody of any one of claims 6-9, wherein the antibody is an scFv antibody; optionally, wherein the scFv antibody comprises an amino acid sequence of at least 90% sequence identity to a sequence selected from SEQ ID NO: 45 and 46.

12. The antibody of any one of the claims 1-11, wherein the antibody is an antibody fragment selected from Fv, scFv, Fab, Fab′, F(ab′)2, and Fab′-SH.

13. The antibody of any one of claims 1-11, wherein the antibody comprises a canine heavy chain constant region selected from a constant region of IgG-A, IgG-B, IgG-C, and IgG-D.

14. The antibody of any one of claims 1-13, wherein the antibody comprises:
(i) a canine light chain constant region and/or a canine heavy chain constant region;
(ii) a feline light chain constant region and/or a feline heavy chain constant region; or
(iii) an equine light chain constant region and/or an equine heavy chain constant region.

15. The antibody of any one of claims 1-14, wherein the antibody comprises a heavy chain constant region with a “Y” mutation at EU Numbering position 252.

16. A bispecific antibody that binds to canine, feline, and/or equine IL23 and canine, feline, and/or equine TNFα, wherein the antibody comprises:
(a) an anti-IL23 light chain (LC) comprising a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6); an anti-IL23 heavy chain (HC) comprising a VH domain having an HVR-H1 region comprising an amino acid sequence KGSGYSFITYWLG (SEQ ID NO: 8), an HVR-H2 region comprising an amino acid sequence IMSPVDSDIR (SEQ ID NO: 9), and an HVR-H3 region comprising an amino acid sequence ARRRPGQGYFDF (SEQ ID NO: 10); and an anti-TNFα scFv antibody fused to the HC, wherein the anti-TNFα scFv antibody comprises a VL domain having an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29); and a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33); or
(b) an anti-TNFα light chain (LC) comprising a VL domain an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29), and an anti-TNFα heavy chain (HC) comprising a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33), and an anti-IL23 scFv antibody fused to the HC, wherein the anti-IL23 scFv antibody comprises a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6), and a VH domain having an HVR-H1 region comprising an amino acid sequence KGSGYSFITYWLG (SEQ ID NO: 8), an HVR-H2 region comprising an amino acid sequence IMSPVDSDIR (SEQ ID NO: 9) and an HVR-H3 region comprising an amino acid sequence ARRRPGQGYFDF (SEQ ID NO: 10).

17. The bispecific antibody of claim 16, wherein:
(a) the anti-IL23 light chain (LC) comprises a light chain variable domain (VL) amino acid sequence having at least 90% identity to a sequence selected from SEQ ID NO: 3, 11, 13, and 15, and the anti-IL23 heavy chain (HC) comprises a heavy chain variable domain (VH) amino acid sequence having at least 90% identity to a sequence selected from SEQ ID NO: 7, 12, 14, and 16, wherein the HC is fused to an anti-TNFα scFv antibody comprising a light chain variable domain (VL) amino acid sequence having at least 90% identity to a sequence selected from SEQ ID NO: 26, 34, and 36 and a heavy chain variable domain (VH) amino acid sequence having at least 90% identity to a sequence selected from SEQ ID NO: 30, 35, and 37; or
(b) the anti-TNFα light chain (LC) comprises a light chain variable domain (VL) amino acid sequence having at least 90% identity to a sequence selected from SEQ ID NO: 26, 34, and 36, and the anti-TNFα heavy chain (HC) comprises a heavy chain variable domain (VH) amino acid sequence having at least 90% identity to a sequence selected from SEQ ID NO: 30, 35, and 37, wherein the HC is fused to an anti-IL23 scFv antibody comprising a light chain variable domain (VL) amino acid sequence having at least 90% identity to a sequence selected from SEQ ID NO: 3, 11, 13, and 15 and a heavy chain variable domain (VH) amino acid sequence having at least 90% identity to a sequence selected from SEQ ID NO: 7, 12, 14, and 16.

18. The bispecific antibody of any one of claims 16-17, wherein the light chain (LC) comprises an amino acid sequence having at least 90% identity to a sequence selected from SEQ ID NO: 19 and 22; and the heavy chain (HC) comprises an amino acid sequence having at least 90% sequence identity to SEQ ID NO: 47 and 48; optionally, wherein the antibody comprises:
(i) the LC amino acid sequence of SEQ ID NO: 19, and the HC amino acid sequence of SEQ ID NO: 47;
(ii) the LC amino acid sequence of SEQ ID NO: 19, and the HC amino acid sequence of SEQ ID NO: 48;
(iii) the LC amino acid sequence of SEQ ID NO: 22, and the HC amino acid sequence of SEQ ID NO: 47; or
(iv) the LC amino acid sequence of SEQ ID NO: 22, and the HC amino acid sequence of SEQ ID NO: 48.

19. A bispecific antibody that binds to canine, feline, and/or equine IL23 and canine, feline, and/or equine TNFα, wherein the antibody comprises:
(i) an anti-IL23 light chain (LC) comprising a VL domain having an HVR-L1 region comprising an amino acid sequence RASQGISSWLA (SEQ ID NO: 4), an HVR-L2 region comprising an amino acid sequence YAASSLQS (SEQ ID NO: 5), and an HVR-L3 region comprising an amino acid sequence QQYNIYPYT (SEQ ID NO: 6);
(ii) an anti-IL23 heavy chain (HC) comprising a VH domain having an HVR-H1 region comprising an amino acid sequence KGSGYSFITYWLG (SEQ ID NO: 8), an HVR-H2 region comprising an amino acid sequence IMSPVDSDIR (SEQ ID NO: 9), and an HVR-H3 region comprising an amino acid sequence ARRRPGQGYFDF (SEQ ID NO: 10);
(iii) an anti-TNFα light chain (LC) comprising a VL domain an HVR-L1 region comprising an amino acid sequence RASQGIRNYLA (SEQ ID NO: 27), an HVR-L2 region comprising an amino acid sequence AASTLQ (SEQ ID NO: 28), and an HVR-L3 comprising an amino acid sequence QRYNRAPYT (SEQ ID NO: 29); and
(iv) an anti-TNFα heavy chain (HC) comprising a VH domain having an HVR-H1 region comprising an amino acid sequence FTFDDYAMH (SEQ ID NO: 31), an HVR-H2 region comprising an amino acid sequence AITWNSGHIDYADSVEGR (SEQ ID NO: 32), and an HVR-H3 region comprising an amino acid sequence AKVSYLSTASSLDY (SEQ ID NO: 33).

20. The bispecific antibody of claim 19, wherein:
(i) the anti-IL23 light chain (LC) comprises a light chain variable domain (VL) amino acid sequence having at least 90% identity to a sequence selected from SEQ ID NO: 3, 11, 13, and 15;
(ii) the anti-IL23 heavy chain (HC) comprises a heavy chain variable domain (VH) amino acid sequence having at least 90% identity to a sequence selected from SEQ ID NO: 7, 12, 14, and 16;
(iii) the anti-TNFα light chain (LC) comprises a light chain variable domain (VL) amino acid sequence having at least 90% identity to a sequence selected from SEQ ID NO: 26, 34, and 36; and
(iv) the anti-TNFα heavy chain (HC) comprises a heavy chain variable domain (VH) amino acid sequence having at least 90% identity to a sequence selected from SEQ ID NO: 30, 35, and 37.

21. The bispecific antibody of any one of claims 19-20, wherein:
(i) the anti-IL23 light chain (LC) comprises an amino acid sequence having at least 90% identity to a sequence selected from SEQ ID NO: 19 and 22;
(ii) the anti-IL23 heavy chain (HC) comprises an amino acid sequence having at least 90% sequence identity to SEQ ID NO: 20, 21, and 23;
(iii) the anti-TNFα light chain (LC) comprises an amino acid sequence having at least 90% identity to a sequence selected from SEQ ID NO: 40 and 41; and
(iv) the anti-TNFα heavy chain (HC) comprises an amino acid sequence having at least 90% sequence identity to SEQ ID NO: 42, 43, and 44.

22. An isolated nucleic acid or vector encoding the antibody of any one of claims 1 to 21.

23. An isolated host cell comprising the nucleic acid or vector of claim 22.

24. A method of producing an antibody comprising culturing the host cell of claim 23 and isolating the antibody.

25. A pharmaceutical composition comprising the antibody of any one of claims 1 to 21 and a pharmaceutically acceptable carrier.

26. A method of treating a canine, feline, or equine having a condition associated with IL23, the method comprising administering to the canine, feline, or equine a therapeutically effective amount of the antibody of any one of claims 1-21 or the pharmaceutical composition of claim 25.

27. A method of maintaining remission of a condition associated with IL23 in a canine, feline, or equine, the method comprising administering to the canine, feline, or equine a therapeutically effective amount of the antibody of any one of claims 1-21 or the pharmaceutical composition of claim 25.

28. The method of any one of claims 26-27, wherein the condition associated with IL23 is an inflammatory disease.

29. The method of any one of claims 26-28, wherein the condition associated with IL23 is a gastrointestinal inflammatory disease.

30. The method of any one of claims 26-29, wherein the condition associated with IL23 is inflammatory bowel disease.

31. The method of any one of claims 26-30, wherein the condition associated with IL23 is ankylosing spondylitis, asthma, cancer, Crohn's disease, idiopathic arthritis, psoriasis, plaque psoriasis, psoriatic arthritis, rheumatoid arthritis, osteoarthritis or ulcerative colitis.

32. A method of treating a canine, feline, or equine having a condition associated with IL23 and TNFα, the method comprising administering to the canine, feline, or equine a therapeutically effective amount of a IL23 antibody of any one of claims 1-5 or 12-21 and a TNFα antibody of any one of claims 6-21, or the pharmaceutical composition of claim 25.

33. A method of treating a canine, feline, or equine having a condition associated with IL23/TNFα, the method comprising administering to the canine, feline, or equine a therapeutically effective amount of a bispecific IL23/TNFα antibody of any one of claims 16-21.

34. The method of any one of claims 26-33, wherein the antibody or the pharmaceutical composition is administered parenterally.

35. The method of any one of claims 26-34, wherein the antibody or the pharmaceutical composition is administered by an intramuscular route, an intraperitoneal route, an intracerebrospinal route, a subcutaneous route, an intra-arterial route, an intrasynovial route, an intrathecal route, or an inhalation route.

36. The method of any one of claims 26-35, wherein the method further comprises administering an IL17 antibody, an IL-5 antibody, an IL-31 antibody, an IL4 antibody, an IL13 antibody, an IL23 antibody, an IgE antibody, a CD11α antibody, an IL6R antibody, an α4-Intergrin antibody, a beta7-Intergrin antibody, an IL12 antibody, an IL1β antibody, or an anti-BlyS antibody.

37. The method of any one of claims 23-33, wherein the antibody is administered at an amount in the range of 0.01 mg/kg body weight to 100 mg/kg body weight per dose.

38. A method of reducing IL23 and/or TNFα signaling function in a cell, the method comprising exposing to the cell the antibody of any one of claims 1-21 under conditions permissive for binding of the antibody to IL23 and/or TNFα, thereby reducing binding to IL23 and/or TNFα signaling function by the cell.

39. The method of claim 38, wherein the cell is exposed to the antibody or the pharmaceutical composition in vivo.

40. The method of any one of claims 38-39, wherein the cell is a canine cell, a feline cell, or an equine cell.

41. A method for detecting IL23 and/or TNFα in a sample from a companion animal species comprising contacting the sample with the antibody of any one of claims 1 to 21 under conditions permissive for binding of the antibody to IL23 and/or TNFα and detecting whether a complex is formed between the antibody and IL23 and/or TNFα in the sample.
